(19) United States
(12) Patent Application Publication
(10) Pub. No.: US 2025/0064843 A1
(43) Pub. Date: Feb. 27, 2025

(54) METHOD AND MEDICAMENT FOR TREATING AMYOTROPHIC LATERAL SCLEROSIS

(71) Applicants: 4B TECHNOLOGIES (BEIJING) CO., LIMITED, Beijing (CN); INSILICO MEDICINE IP LIMITED, Hong Kong (CN)

(72) Inventors: Bai Lu, Beijing (CN); Xiaoming Guan, Jiangsu (CN); Zavoronkovs Aleksandrs, Hong Kong (CN); Wing Frank Pun, Hong Kong (CN); Xi Long, Chongqing (CN); Hei Man Liu, Hong Kong (CN); Ju Wang, Shanghai (CN); Feng Ren, Shanghai (CN)

(21) Appl. No.: 18/721,283

(22) PCT Filed: Dec. 20, 2022
(86) PCT No.: PCT/CN2022/140288

§ 371 Date: Jun. 18, 2024

(30) Foreign Application Priority Data
Dec. 20, 2021 (WO) PCT/CN2021/139736

(51) Int. Cl. A61K 31/706; A61K 31/203; A61K 31/343; A61K 31/4409; A61K 31/4439; A61K 31/519; A61K 31/55; A61K 31/567; A61K 38/13

(52) U.S. Cl. CPC A61K 31/706; A61K 31/203; A61K 31/343; A61K 31/4409; A61K 31/4439; A61K 31/519; A61K 31/55; A61K 31/567; A61K 38/13

(57) ABSTRACT

Provided is a method and a medicament for treating amyotrophic lateral sclerosis (ALS) in a subject in need thereof, comprising administering to the subject an effective amount of one or more active agents selected from the group consisting of a retinoic acid receptor alpha (RARα) agonist, a voltage-gated potassium channel (KCNB2) inhibitor, an adrenergic receptor α2B (ADRA2B) antagonist, a DNA methyltransferase 3 alpha (DNMT3A) antagonist, an insulin like growth factor 1 receptor (IGF1R) inhibitor, a mitogen-activated protein kinase 1 (MAPK1) inhibitor, a nitric oxide synthase 1 (NOS1) inhibitor, a glucocorticoid receptors (NR3C1) antagonist, and a peptidylprolyl Isomerase A (PPIA) antagonist.

### CROSS-REFERENCE TO RELATED APPLICATIONS

The present application claims priority to PCT/CN2021/139736 entitled “METHOD AND MEDICAMENT FOR TREATING AMYOTROPHIC LATERAL SCLEROSIS” filed on Dec. 20, 2021, which is incorporated herein by reference in its entirety.

### FIELD

The present invention relates to the field of medicine, in particular, to a method for treating amyotrophic lateral sclerosis (ALS).

### BACKGROUND

Amyotrophic lateral sclerosis (ALS) is a rare neuromuscular disease caused by the degeneration of motor neurons (MNs) in the brain and spinal cord. It is the most common MN disease, with the incidence ranging from 0.6 to 3.8 per 100,000 person-years[1]. Approximately 16,500 persons were diagnosed with ALS in the United States in 2015. The onset of the disease is typically in middle adulthood, with mean survival time in the range of 3-5 years after diagnosis. Although the signs and symptoms of ALS vary due to the difference in the region of neurons being affected, patients usually experience painless progressive muscle weakness and paralysis.

Despite several strategies that have been proposed to classify ALS, the majority of studies categorizes the disease based on its root causes-familial or sporadic. Familial ALS contributes to 10% of the cases and involves mutations in specific genetic loci that are inherited in an autosomal dominant manner. Over 20 genetic risk factors were identified for fALS. Notably, SODI, TARDBP, C9orf72, and FUS have been extensively characterized. According to a pooled summary of mutation frequency in 111 studies, those four major ALS-associated genes could explain 47.7% fALS and 5.2% sALS cases[2], leaving a substantial fraction of the genetic basis of ALS, especially sALS (over 90% of ALS cases), undiscovered. Given the genetic involvement in ALS is heterogeneous, several pathophysiological mechanisms have been hypothesized, including aberrant proteostasis, altered RNA metabolism, nucleocytoplasmic transport defects, mitochondrial dysfunction, DNA repair defects, axonal transport defects, vesicle transport dysregulation, excitotoxicity, oligodendrocyte dysfunction, and neuroinflammation.

Till now, ALS remains an incurable disease due to an inadequate understanding of disease mechanisms. FDA has approved four drugs for the treatment of ALS, including Riluzole, Edaravone, Tiglutik (thickened Riluzole), and Nuedexta. Riluzole—an inhibitor of sodium channel α subunit—is the first FDA-approved neuroprotective agent for ALS and the only drug that prolongs the survival of ALS patients. It functions as an anti-excitotoxic agent and G protein-coupled receptor signaling activator[3]. Unfortunately, riluzole offers limited benefit to the patients by extending survival for 2-3 months. Edaravone is a potent antioxidant that protects nerve cells against reactive oxygen species-induced death. Multiple clinical studies have illustrated its effect on slowing disease progression in small subsets of ALS patients.

### SUMMARY

The purpose of the present invention is to provide a therapy or combination therapy for the treatment of amyotrophic lateral sclerosis, as well as drug combinations and kits.

One aspect of the present invention provides a method for treating amyotrophic lateral sclerosis (ALS) in a subject in need thereof, the method comprises administering to the subject an effective amount of one or more active agents selected from the group consisting of a retinoic acid receptor alpha (RARα) agonist, a voltage-gated potassium channel (KCNB2) inhibitor, an adrenergic receptor α2B (ADRA2B) antagonist, a DNA methyltransferase 3 alpha (DNMT3A) antagonist, an insulin like growth factor 1 receptor (IGF1R) inhibitor, a mitogen-activated protein kinase 1 (MAPK1) inhibitor, a nitric oxide synthase 1 (NOS1) inhibitor, a glucocorticoid receptors (NR3C1) antagonist, and a peptidylprolyl Isomerase A (PPIA) antagonist.

In some embodiments, the retinoic acid receptor alpha (RARα) agonist is selected from acitretin and derivative thereof.

In some embodiments, the voltage-gated potassium channel (KCNB2) inhibitor is selected from dalfampridine and derivative thereof.

In some embodiments, the adrenergic receptor a 2B (ADRA2B) antagonist is selected from mirtazapine and derivative thereof.

In some embodiments, the DNA methyltransferase 3 alpha (DNMT3A) antagonist is selected from azacitidine, decitabine and derivatives thereof.

In some embodiments, the insulin like growth factor 1 receptor (IGF1R) inhibitor is selected from AXL-1717 and derivative thereof.

In some embodiments, the mitogen-activated protein kinase 1 (MAPK1) inhibitor is selected from ulixertinib and derivative thereof.

In some embodiments, the nitric oxide synthase 1 (NOS1) inhibitor is selected from ronopterin and derivative thereof.

In some embodiments, the glucocorticoid receptors (NR3C1) antagonist is selected from mifepristones, ORG-34517 and derivatives thereof.

In some embodiments, the peptidylprolyl Isomerase A (PPIA) antagonist is selected from cyclosporine and derivative thereof.

In some embodiments, the method comprises administering to the subject an effective amount of a first active agent and a second active agent, wherein the first active agent and the second active agent are different from each other and are independently selected from the group consisting of the retinoic acid receptor alpha (RARα) agonist, the voltage-gated potassium channel (KCNB2) inhibitor, the adrenergic receptor α2B (ADRA2B) antagonist, the DNA methyltransferase 3 alpha (DNMT3A) antagonist, the insulin like growth factor 1 receptor (IGF1R) inhibitor, the mitogen-activated protein kinase 1 (MAPK1) inhibitor, the nitric oxide synthase 1 (NOS1) inhibitor, the glucocorticoid receptors (NR3C1) antagonist and the peptidylprolyl Isomerase A (PPIA) antagonist.

In some embodiments, the first active agent is the retinoic acid receptor alpha (RARα) agonist and the second active agent is the voltage-gated potassium channel (KCNB2) inhibitor.

In some embodiments, the first active agent is the retinoic acid receptor alpha (RARα) agonist and the second active agent is the adrenergic receptor α2B (ADRA2B) antagonist.

In some embodiments, the first active agent is the voltage-gated potassium channel (KCNB2) inhibitor and the second active agent is the adrenergic receptor α2B (ADRA2B) antagonist.

In some embodiments, the first active agent and the second active agent are administered simultaneously, separately or sequentially.

In some embodiments, the method comprises administering to the subject an effective amount of a first active agent, a second active agent and a third active agent, wherein the first active agent, the second active agent and the third active agent are different from each other and are independently selected from the group consisting of the retinoic acid receptor alpha (RARα) agonist, the voltage-gated potassium channel (KCNB2) inhibitor, the adrenergic receptor α2B (ADRA2B) antagonist, the DNA methyltransferase 3 alpha (DNMT3A) antagonist, the insulin like growth factor 1 receptor (IGF1R) inhibitor, the mitogen-activated protein kinase 1 (MAPK1) inhibitor, the nitric oxide synthase 1 (NOS1) inhibitor, the glucocorticoid receptors (NR3C1) antagonist and the peptidylprolyl Isomerase A (PPIA) antagonist.

In some embodiments, the first active agent is the retinoic acid receptor alpha (RARα) agonist, the second active agent is the voltage-gated potassium channel (KCNB2) inhibitor and the third active agent is the adrenergic receptor α2B (ADRA2B) antagonist.

In some embodiments, the first active agent, the second active agent and the third active agent are administered simultaneously, separately or sequentially.

Another aspect of the present invention provides a combination of two or more active agents selected from the group consisting of a retinoic acid receptor alpha (RARα) agonist, a voltage-gated potassium channel (KCNB2) inhibitor, an adrenergic receptor α2B (ADRA2B) antagonist, a DNA methyltransferase 3 alpha (DNMT3A) antagonist, an insulin like growth factor 1 receptor (IGFIR) inhibitor, a mitogen-activated protein kinase 1 (MAPK1) inhibitor, a nitric oxide synthase 1 (NOS1) inhibitor, a glucocorticoid receptors (NR3C1) antagonist, and a peptidylprolyl Isomerase A (PPIA) antagonist; wherein the combination is used for the treatment of amyotrophic lateral sclerosis.

In some embodiments, the retinoic acid receptor alpha (RARα) agonist is selected from acitretin and derivative thereof.

In some embodiments, the voltage-gated potassium channel (KCNB2) inhibitor is selected from dalfampridine and derivative thereof.

In some embodiments, the adrenergic receptor α2B (ADRA2B) antagonist is selected from mirtazapine and derivative thereof.

In some embodiments, the DNA methyltransferase 3 alpha (DNMT3A) antagonist is selected from azacitidine, decitabine and derivatives thereof.

In some embodiments, the insulin like growth factor 1 receptor (IGF1R) inhibitor is selected from AXL-1717 and derivative thereof.

In some embodiments, the mitogen-activated protein kinase 1 (MAPK1) inhibitor is selected from ulixertinib and derivative thereof.

In some embodiments, the nitric oxide synthase 1 (NOS1) inhibitor is selected from ronopterin and derivative thereof.

In some embodiments, the glucocorticoid receptors (NR3C1) antagonist is selected from mifepristones, ORG-34517 and derivatives thereof.

In some embodiments, the peptidylprolyl Isomerase A (PPIA) antagonist is selected from cyclosporine and derivative thereof.

In some embodiments, the combination comprises a first active agent and a second active agent, wherein the first active agent and the second active agent are different from each other and are independently selected from the group consisting of the retinoic acid receptor alpha (RARα) agonist, the voltage-gated potassium channel (KCNB2) inhibitor, the adrenergic receptor α2B (ADRA2B) antagonist, the DNA methyltransferase 3 alpha (DNMT3A) antagonist, the insulin like growth factor 1 receptor (IGF1R) inhibitor, the mitogen-activated protein kinase 1 (MAPK1) inhibitor, the nitric oxide synthase 1 (NOS1) inhibitor, the glucocorticoid receptors (NR3C1) antagonist and the peptidylprolyl Isomerase A (PPIA) antagonist.

In some embodiments, the first active agent is the retinoic acid receptor alpha (RARα) agonist and the second active agent is the voltage-gated potassium channel (KCNB2) inhibitor.

In some embodiments, the first active agent is the retinoic acid receptor alpha (RARα) agonist and the second active agent is the adrenergic receptor α2B (ADRA2B) antagonist.

In some embodiments, the first active agent is the voltage-gated potassium channel (KCNB2) inhibitor and the second active agent is the adrenergic receptor α2B (ADRA2B) antagonist.

In some embodiments, the combination comprises a first active agent, a second active agent and a third active agent, wherein the first active agent, the second active agent and the third active agent are different from each other and are independently selected from the group consisting of the retinoic acid receptor alpha (RARα) agonist, the voltage-gated potassium channel (KCNB2) inhibitor, the adrenergic receptor α2B (ADRA2B) antagonist, the DNA methyltransferase 3 alpha (DNMT3A) antagonist, the insulin like growth factor 1 receptor (IGF1R) inhibitor, the mitogen-activated protein kinase 1 (MAPK1) inhibitor, the nitric oxide synthase 1 (NOS1) inhibitor, the glucocorticoid receptors (NR3C1) antagonist and the peptidylprolyl Isomerase A (PPIA) antagonist.

In some embodiments, the first active agent is the retinoic acid receptor alpha (RARα) agonist, the second active agent is the voltage-gated potassium channel (KCNB2) inhibitor and the third active agent is the adrenergic receptor α2B (ADRA2B) antagonist.

Another aspect of the present invention provides a pharmaceutical composition comprising any one of the above-mentioned combinations and a pharmaceutically acceptable carrier, wherein the combination may be used for the treatment of amyotrophic lateral sclerosis.

Another aspect of the present invention provides a kit comprising any one of the above-mentioned combinations or the above-mentioned pharmaceutical composition and an instruction for use, wherein the instruction describes the use of the combination or the pharmaceutical composition for treating amyotrophic lateral sclerosis.

In some embodiments, in the kit, the two or more active agents are contained in the same or separate containers.

Another aspect of the present invention provides use of one or more active agents selected from the group consisting of a retinoic acid receptor alpha (RARα) agonist, a voltage-gated potassium channel (KCNB2) inhibitor, an adrenergic receptor α2B (ADRA2B) antagonist, a DNA methyltransferase 3 alpha (DNMT3A) antagonist, an insulin like growth factor 1 receptor (IGF1R) inhibitor, a mitogen-activated protein kinase 1 (MAPK1) inhibitor, a nitric oxide synthase 1 (NOS1) inhibitor, a glucocorticoid receptors (NR3C1) antagonist, and a peptidylprolyl Isomerase A (PPIA) antagonist in the manufacture of a medicament for treating amyotrophic lateral sclerosis.

In some embodiments, the retinoic acid receptor alpha (RARα) agonist is selected from acitretin and derivative thereof.

In some embodiments, the voltage-gated potassium channel (KCNB2) inhibitor is selected from dalfampridine and derivative thereof.

In some embodiments, the adrenergic receptor α2B (ADRA2B) antagonist is selected from mirtazapine and derivative thereof.

In some embodiments, the DNA methyltransferase 3 alpha (DNMT3A) antagonist is selected from azacitidine, decitabine and derivatives thereof.

In some embodiments, the insulin like growth factor 1 receptor (IGF1R) inhibitor is selected from AXL-1717 and derivative thereof.

In some embodiments, the mitogen-activated protein kinase 1 (MAPK1) inhibitor is selected from ulixertinib and derivative thereof.

In some embodiments, the nitric oxide synthase 1 (NOS1) inhibitor is selected from ronopterin and derivative thereof.

In some embodiments, the glucocorticoid receptors (NR3C1) antagonist is selected from mifepristones, ORG-34517 and derivatives thereof.

In some embodiments, the peptidylprolyl Isomerase A (PPIA) antagonist is selected from cyclosporine and derivative thereof.

In some embodiments, said one or more active agents comprise a combination of a first active agent and a second active agent, wherein the first active agent and the second active agent are different from each other and are independently selected from the group consisting of the retinoic acid receptor alpha (RARα) agonist, the voltage-gated potassium channel (KCNB2) inhibitor, the adrenergic receptor α2B (ADRA2B) antagonist, the DNA methyltransferase 3 alpha (DNMT3A) antagonist, the insulin like growth factor 1 receptor (IGF1R) inhibitor, the mitogen-activated protein kinase 1 (MAPK1) inhibitor, the nitric oxide synthase 1 (NOS1) inhibitor, the glucocorticoid receptors (NR3C1) antagonist and the peptidylprolyl Isomerase A (PPIA) antagonist.

In some embodiments, the first active agent is the retinoic acid receptor alpha (RARα) agonist and the second active agent is the voltage-gated potassium channel (KCNB2) inhibitor.

In some embodiments, the first active agent is the retinoic acid receptor alpha (RARa) agonist and the second active agent is the adrenergic receptor α2B (ADRA2B) antagonist.

In some embodiments, the first active agent is the voltage-gated potassium channel (KCNB2) inhibitor and the second active agent is the adrenergic receptor a 2B (ADRA2B) antagonist.

In some embodiments, said one or more active agents comprise a combination of a first active agent, a second active agent and a third active agent, wherein the first active agent, the second active agent and the third active agent are different from each other and are selected from the group consisting of the retinoic acid receptor alpha (RARα) agonist, the voltage-gated potassium channel (KCNB2) inhibitor, the adrenergic receptor α2B (ADRA2B) antagonist, the DNA methyltransferase 3 alpha (DNMT3A) antagonist, the insulin like growth factor 1 receptor (IGFIR) inhibitor, the mitogen-activated protein kinase 1(MAPK1) inhibitor, the nitric oxide synthase 1 (NOS1) inhibitor, the glucocorticoid receptors (NR3C1) antagonist and the peptidylprolyl Isomerase A (PPIA) antagonist.

In some embodiments, the first active agent is the retinoic acid receptor alpha (RARα) agonist, the second active agent is the voltage-gated potassium channel (KCNB2) inhibitor and the third active agent is the adrenergic receptor α2B (ADRA2B) antagonist.

Another aspect of the present invention provides a medicament for use in the treatment of amyotrophic lateral sclerosis, wherein the medicament comprises one or more active agents selected from the group consisting of a retinoic acid receptor alpha (RARα) agonist, a voltage-gated potassium channel (KCNB2) inhibitor, an adrenergic receptor α2B (ADRA2B) antagonist, a DNA methyltransferase 3 alpha (DNMT3A) antagonist, an insulin like growth factor 1 receptor (IGF1R) inhibitor, a mitogen-activated protein kinase 1(MAPK1) inhibitor, a nitric oxide synthase 1 (NOS1) inhibitor, a glucocorticoid receptors (NR3C1) antagonist, and a peptidylprolyl Isomerase A (PPIA) antagonist.

In some embodiments, the retinoic acid receptor alpha (RARα) agonist is selected from acitretin and derivative thereof.

In some embodiments, the voltage-gated potassium channel (KCNB2) inhibitor is selected from dalfampridine and derivative thereof.

In some embodiments, the adrenergic receptor α2B (ADRA2B) antagonist is selected from mirtazapine and derivative thereof.

In some embodiments, the DNA methyltransferase 3 alpha (DNMT3A) antagonist is selected from azacitidine, decitabine and derivatives thereof.

In some embodiments, the insulin like growth factor 1 receptor (IGF1R) inhibitor is selected from AXL-1717 and derivative thereof.

In some embodiments, the mitogen-activated protein kinase 1 (MAPK1) inhibitor is selected from ulixertinib and derivative thereof.

In some embodiments, the nitric oxide synthase 1 (NOS1) inhibitor is selected from ronopterin and derivative thereof.

In some embodiments, the glucocorticoid receptors (NR3C1) antagonist is selected from mifepristones, ORG-34517 and derivatives thereof.

In some embodiments, the peptidylprolyl Isomerase A (PPIA) antagonist is selected from cyclosporine and derivative thereof.

In some embodiments, the medicament comprises a combination of a first active agent and a second active agent, wherein the first active agent and the second active agent are different from each other and are selected from the group consisting of the retinoic acid receptor alpha (RARα) agonist, the voltage-gated potassium channel (KCNB2) inhibitor, the adrenergic receptor α2B (ADRA2B) antagonist, the DNA methyltransferase 3 alpha (DNMT3A) antagonist, the insulin like growth factor 1 receptor (IGF1R) inhibitor, the mitogen-activated protein kinase 1 (MAPK1) inhibitor, the nitric oxide synthase 1 (NOS1) inhibitor, the glucocorticoid receptors (NR3C1) antagonist and the peptidylprolyl Isomerase A (PPIA) antagonist.

In some embodiments, the first active agent is the retinoic acid receptor alpha (RARα) agonist and the second active agent is the voltage-gated potassium channel (KCNB2) inhibitor.

In some embodiments, the first active agent is the retinoic acid receptor alpha (RARα) agonist and the second active agent is the adrenergic receptor α2B (ADRA2B) antagonist.

In some embodiments, the first active agent is the voltage-gated potassium channel (KCNB2) inhibitor and the second active agent is the adrenergic receptor α2B (ADRA2B) antagonist.

In some embodiments, the medicament comprises a combination of a first active agent, a second active agent and a third active agent, wherein the first active agent, the second active agent and the third active agent are different from each other and are selected from the group consisting of the retinoic acid receptor alpha (RARα) agonist, the voltage-gated potassium channel (KCNB2) inhibitor, the adrenergic receptor α2B (ADRA2B) antagonist, the DNA methyltransferase 3 alpha (DNMT3A) antagonist, the insulin like growth factor 1 receptor (IGF1R) inhibitor, the mitogen-activated protein kinase 1 (MAPK1) inhibitor, the nitric oxide synthase 1 (NOS1) inhibitor, the glucocorticoid receptors (NR3C1) antagonist and the peptidylprolyl Isomerase A (PPIA) antagonist.

In some embodiments, the first active agent is the retinoic acid receptor alpha (RARα) agonist, the second active agent is the voltage-gated potassium channel (KCNB2) inhibitor and the third active agent is the adrenergic receptor α2B (ADRA2B) antagonist.

### BRIEF DESCRIPTION OF THE DRAWINGS

FIG. 1 shows ALS-related datasets in PandaOmics™.

FIG. 2 shows filter setting for high-confidence targets identification.

FIG. 3 shows filter setting for novel targets identification.

FIG. 4 shows schematic representation of scoring-approach validation.

FIG. 5 shows network of dysregulated pathways in CNS comparisons. Each node represents a dysregulated pathway consisting of a set of genes. Nodes with similar gene contents (similarity coefficient >0.35) were connected by edges, and the thickness of node-linking edges is proportional to the similarity between a pair of gene sets. Clusters of pathways were annotated based on the hierarchical level of pathways retrieved from the Reactome database.

FIG. 6 shows network of dysregulated pathways in diMN comparisons. Notations refer to FIG. 5.

FIG. 7 shows flowchart for ALS target discovery and drug repurposing.

### DETAILED DESCRIPTION

It should be understood that this invention is not limited to particular embodiments described herein. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are to disclose and describe the methods and/or materials in connection with which the publications are cited.

Where a range of values with one or two limits is provided, it is understood that a smaller range between any stated intervening value in that stated range and either limit of that stated range is encompassed within the invention. Where the stated range includes one or two limits, ranges excluding either or both of the limits are also included in the invention.

### Terminology

It must be noted that as used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Unless otherwise stated, the term “comprise”, “include”, “contain” and variations of these terms, such as comprising, comprises and comprised, are not intended to exclude further members, components, integers or steps. These terms also encompass the meaning of “consist of” or “consisting of”. The term “consist of” or “consisting of” is a particular embodiment of the term “comprise”, wherein any other non-stated member, component, integer or step is excluded.

The term “about” refers to a range equal to the particular value plus or minus ten percent (+/−10%).

The term “and/or” refers to any one, several or all of the elements connected by the term.

The term “amyotrophic lateral sclerosis (ALS)” is also called classical motor neuron disease (MND), which is a progressive fatal neuromuscular disorder that is characterized by weakness, muscle wasting, and fasciculations (increased reflexes). Cognitive function is retained except where ALS is associated with dementia. The disease primarily affects motor neurons and is characterized by progressive degeneration of the motor neurons in the cerebral cortex, brainstem nuclei and anterior horns of the spinal cord. Individuals afflicted by the disease exhibit weakness of limbs and difficulty in speech and swallowing.

The term “treat”, “treating” or “treatment”, as used herein, refers to alleviating, inhibiting and/or reversing the progress of a disease (such as ALS). The term “treating” is inclusive of any indicia of success in the treatment or amelioration of the disease, including any objective or subjective parameter such as abatement; remission; diminishing of symptoms or making the injury, pathology or condition more tolerable to the subject; delaying or slowing in the rate of progression, etc. Measurement of the treatment or amelioration may be based on, e.g., the results of a physical examination, a pathological test and/or a diagnostic test as known in the art. Treating may also refer to reducing the incidence or onset of a disease, or a recurrence thereof (such as a lengthening in time of remission), as compared to that which would occur in the absence of the measure taken. Clinically, such a treatment can also be called prevention.

The term “active agent”, as used herein, refers to a pharmaceutically active chemical that provides some pharmacologic effect and is used for treating or preventing a disease, such as ALS.

The term “inhibitor” and “antagonist”, as used herein, can be used interchangeably and refer to any molecule that partially or fully blocks or inhibits an activity of a target (such as the protein used as a target in the present invention). In a similar manner, the term “agonist” refers to any molecule that stimulates, activates, enhances an activity of a target (such as the protein used as a target in the present invention).

The term “derivative” of a compound, as used herein, refers to any pharmaceutically acceptable molecule that is derived from (i.e., structurally related to) the compound and has similar or substantially the same activity as the compound, which upon administration to a subject is capable of providing (directly or indirectly) a compound of the active agent or an active metabolite thereof. Examples of the derivatives include, but are not limited to, pharmaceutically acceptable salt, hydrate, solvate, prodrug or metabolite.

The term “pharmaceutically acceptable salt”, as used herein, refers to a relatively nontoxic, inorganic or organic acid salt of a compound of the invention. These salts may be prepared in situ during the final isolation and purification of the compounds or by reacting the purified compound in its free form separately with a suitable organic or inorganic acid and isolating the salt thus formed. Representative acid salts include, but are not limited to, acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinafoate salts. In one embodiment, the pharmaceutically acceptable salt is a hydrochloride/chloride salt.

The term “solvate”, as used herein, refers to a complex of variable stoichiometry formed by a solute (e.g., the active agent of the present invention) and a solvent. Such solvents for the purpose of the invention may not interfere with the biological activity of the solute. Examples of suitable solvents include, but are not limited to, water, methanol, ethanol and acetic acid.

The term “prodrug” of a compound, as used herein, refers to a precursor, which when administered to a biological system, generates said compound as a result. For example, prodrugs can have the structure X-drug wherein X is an inert carrier moiety and drug is the active compound,

The term “metabolite” of a compound, as used herein, refers to a molecule which results from a modification or processing of the compound after administration to a subject. The term “metabolite” may designate a modified or processed drug that retains at least part of the activity of the parent compound.

The term “combination”, as used herein, refers to two or more active agents and/or therapies which may be administered simultaneously or sequentially, in a single dosage form or separately. The two or more active agents and/or therapies in a combination may be administered through different routes and protocols. The two or more active agents in a combination may be formulated together or separately.

The term “simultaneous” or “simultaneously”, as used herein, means the administration of each of the two or more active agents to a subject at the same time. The two or more active agents may be formulated into a single dosage form (fixed dose combination) or separate dosage forms (non-fixed).

The term “separate” or “separately”, as used herein, means the administration of each of the two or more active agents to a subject from separate (non-fixed) dosage forms simultaneously or sequentially in any order. There may be a specified time interval for administration of each active agent.

The term “sequential” or “sequentially”, as used herein, means the administration of each of the two or more active agents to a subject from separate (non-fixed) dosage forms in separate actions. The two administration actions may be linked by a specified time interval. The term “pharmaceutical composition”, as used herein, can be used interchangeably with “pharmaceutical formulation” or “formulation” and refers to a formulation comprising at least one active agent and at least one pharmaceutically acceptable carrier.

The term “pharmaceutically acceptable”, as used herein, refers to those compounds, materials, compositions, and/or dosage forms which are, within the scope of sound medical judgment, suitable for contact with the tissues of a subject without excessive toxicity, irritation, allergic response, or other problem complications commensurate with a reasonable benefit/risk ratio.

The term “pharmacologically acceptable carrier”, as used herein, refers to any carrier that has substantially no long term or permanent detrimental effect when administered to a subject, such as a stabilizer, diluent, additive, auxiliary, excipient and the like. “Pharmaceutically acceptable carrier” should be a pharmaceutically inert material that has substantially no biological activity and constitutes a substantial part of the formulation.

The term “subject”, as used herein, refers to any organism to which the active agent of the composition of the present invention may be administered, e.g., for experimental, diagnostic, prophylactic, and/or therapeutic purposes. Typical subjects include animals (e.g., mammals such as mice, rats, rabbits, non-human primates such as chimpanzees and other apes and monkey species, and humans). The subject may be a mammal, particularly a human, including a male or female, and including a neonatal, infant, juvenile, adolescent, adult or geriatric, and further is inclusive of various races and ethnicities.

The term “therapeutically effective dose” or “effective dose”, as used herein, which can be used interchangeably with “therapeutically effective amount” or “effective amount”, refers to an amount that is effective for treating a disease (such as ALS) as noted through clinical testing and evaluation, patient observation, and/or the like. An “effective amount” can further designate an amount that causes a detectable change in biological or chemical activity. The detectable changes may be detected and/or further quantified by one skilled in the art for the relevant mechanism or process. Moreover, an “effective amount” can designate an amount that maintains a desired physiological state, i.e., reduces or prevents significant decline and/or promotes improvement in the condition.

The term “unit dosage form”, as used herein, refers to physically discrete units (such as capsules, tablets, or loaded syringe cylinders) suitable as unitary dosages for a subject, each unit containing a predetermined quantity of active agent calculated to produce the desired therapeutic effect, in association with the required pharmaceutical carrier.

The term “unit dose”, as used herein, refers to a dose of a substance (such as an active agent of the present invention) in a unit dosage form.

### Active Agents for Treating ALS

The inventor discovered through extensive research several targets for treating ALS, including retinoic acid receptor alpha (RARα), voltage-gated potassium channel (KCNB2), adrenergic receptor α2B (ADRA2B), DNA methyltransferase 3 alpha (DNMT3A), insulin like growth factor 1 receptor (IGF1R), mitogen-activated protein kinase 1 (MAPK1), nitric oxide synthase 1 (NOS1), glucocorticoid receptors (NR3C1), and peptidylprolyl Isomerase A (PPIA). An agonist or antagonist of one or more of these targets may be used to treat ALS.

The active agent used to treat ALS can be any one selected from the group consisting of a retinoic acid receptor alpha (RARα) agonist, a voltage-gated potassium channel (KCNB2) inhibitor, an adrenergic receptor α2B (ADRA2B) antagonist, a DNA methyltransferase 3 alpha (DNMT3A) antagonist, an insulin like growth factor 1 receptor (IGF1R) inhibitor, a mitogen-activated protein kinase 1 (MAPK1) inhibitor, a nitric oxide synthase 1 (NOS1) inhibitor, a glucocorticoid receptors (NR3C1) antagonist, and a peptidylprolyl Isomerase A (PPIA) antagonist.

Any one of the above active agents can be used alone or in combination with any one or several other active agents. A combination of active agents may include two, three, four, five or more active agents of the present invention. When two or more active agents are used in combination, they may be administered together or separately, at the same time or sequentially. The two or more active agents in a combination may be administered through different routes and protocols. The two or more active agents in a combination may be formulated together to a single dosage form or be formulated separately to two or more separate formulations (for example, each active agent is formulated to an individual pharmaceutical composition having the same or different dosage form). Any one or any combination of two or more of active agents of the present invention can be administered as the pure chemical or be formulated to a pharmaceutical composition before administration. The medicament of the present invention may comprise any one or a combination of two or more active agents of the present invention.

Retinoic acid receptor alpha (RARα), also known as NR1B1 (nuclear receptor subfamily 1, group B, member 1) is a nuclear receptor a transcriptional activator in the retinoid signaling pathway that in humans is encoded by the RARA gene. A preferred agonist of RARαis acitretin. Acitretin is (2E,4E,6E,8E)-9-(4-methoxy-2,3,6-trimethylphenyl)-3,7-dimethylnona-2,4,6,8-tetraenoic acid, which is sold under the trade name Soriatane® or Neotigason®. Acitretin is a synthetic aromatic analogue of retinoic acid (Vitamin A derivative and an active metabolite of etretinate. Acitretin is an agonist of retinoic acid receptors. Potassium voltage-gated channel subfamily B member 2 (KCNB2) belongs to a family of voltage-gated potassium channel and is encoded by the KONB2 gene in humans. inhibitors of potassium voltage-gated channel subfamily B member 2 (KCNB2) include, but are not limited to, dalfampridine. Dalfampridine is the United States Adopted Name (USAN) for the chemical 4-aminopyridine (4-AP), which is a potassium channel blocker. Dalfampridine is sold under the trade name Ampyra®.

Adrenergic receptor α2B (ADRA2B) is a G-protein coupled receptor. It is a subtype of the adrenergic receptor family and is encoded by the ADRA2B gene in humans. Antagonists of adrenergic receptor α2B (ADRA2B) include, but are not limited to, mirtazapine. Mirtazapine is 1,2,3,4,10,14b-hexahydro-2-methylpyrazino [2,1-2]pyrido [2,3-c][2] benzazepine and is sold under the name of Remeron® or RemeronSolTab®. Mirtazapine functions as a strong antagonist of serotonin receptors and adrenergic receptors, including ADRA2B.

DNA methyltransferase 3 alpha (DNMT3A) is an enzyme that catalyzes the transfer of methyl groups to specific CpG structures in DNA, a process called DNA methylation and is encoded by DNMT3A gene in humans. Antagonists of DNA methyltransferase 3 alpha (DNMT3A) include, but are not limited to, azacitidine and decitabine. Azacitidine is 5-azacytidine and is sold under the trade name Vidaza®. Decitabine is 5-aza-2′-deoxycytidine and is sold under the trade name Dacogen®.

Insulin like growth factor 1 receptor (IGF1R) is a transmembrane receptor that is activated by a hormone called insulin-like growth factor 1 (IGF-1) and by a related hormone called IGF-2. It belongs to the large class of tyrosine kinase receptors. Insulin like growth factor 1 receptor (IGF1R) is encoded by IGF1R gene in humans. Antagonists of insulin like growth factor 1 receptor (IGF1R) include, but are not limited to, AXL-1717, which is also called picropodophyllin with a CAS No. 477-47-4.

Mitogen-activated protein kinase 1 (MAPK1) is a member of the MAP kinase family and is encoded by MAPK1 gene in humans. Antagonists of mitogen-activated protein kinase 1 (MAPK1) include, but are not limited to, ulixertinib. Ulixertinib is also called BVD-523 or VRT752271, and has a CAS No. 869886-67-9.

Nitric oxide synthase 1 (NOS1) is also known as neuronal nitric oxide synthase (nNOS). NOSI synthesizes nitric oxide (NO) from L-arginine and is encoded by the NOSI gene in humans. Antagonist of nitric oxide synthase 1 (NOS1) include, but are not limited to, ronopterin. Ronopterin is also called VAS203 and has a CAS No. 206885-38-3.

Nuclear receptor subfamily 3 group C member 1 (NR3C1) is also known as glucocorticoid receptor and is the receptor to which cortisol and other glucocorticoids bind. Nuclear receptor subfamily 3 group C member 1 (NR3C1) is encoded by the NR3C1 gene in humans. Antagonists of NR3C1 include, but are not limited to, mifepristone and ORG-34517.Mifepristone is also known as RU-486, which is 11β-[p-(dimethylamino) phenyl]-17α-(1-propynyl)estra-4,9-dien-17β-ol-3-one. Mifepristone is sold under the trade name Mifegyne®. ORG-34517 has a CAS No. 189035-07-2.

Peptidylprolyl isomerase A (PPIA) is also known as cyclophilin A (CypA) or rotamase A and is encoded by the PPIA gene in humans. Antagonists of peptidylprolyl isomerase A (PPIA) include, but are not limited to, cyclosporine. Cyclosporine, also spelled cyclosporine and cyclosporin, is a calcineurin inhibitor and is sold under the trade name Neoral®, Sandimmune® or Restasis®.

The amount of each of the active agents in a unit dosage form may be 1-1000 mg, such as 1, 2, 3, 4, 5, 6, 7, 8, 9, 10, 11, 12, 13, 14, 15, 16, 17, 18, 19, 20, 25, 30, 35, 40, 50, 60, 70, 75, 80, 90, 100, 110, 120, 125, 130, 140, 150, 160, 170, 175, 180, 190, 200, 250, 300, 350, 400, 450, 500, 600, 700, 750, 800, 900, 1000 mg or any range between any two of the above specific values.

In some embodiments, a combination of two active agents (a first active agent and a second active agent) is used to treat ALS. The first active agent and the second active agent are different from each other. The first active agent and the second active agent are select from the group consisting of a retinoic acid receptor alpha (RARα) agonist, a voltage-gated potassium channel (KCNB2) inhibitor, an adrenergic receptor α2B (ADRA2B) antagonist, a DNA methyltransferase 3 alpha (DNMT3A) antagonist, an insulin like growth factor 1 receptor (IGF1R) inhibitor, a mitogen-activated protein kinase 1 (MAPK1) inhibitor, a nitric oxide synthase 1 (NOS1) inhibitor, a glucocorticoid receptors (NR3C1) antagonist, and a peptidylprolyl Isomerase A (PPIA) antagonist. The two active agents may be formulated in a single dosage form or separately.

In some embodiments, the first active agent may be the retinoic acid receptor alpha (RARα) agonist and the second active agent may be other active agent, such as the voltage-gated potassium channel (KCNB2) inhibitor or the adrenergic receptor α2B (ADRA2B) antagonist. In some embodiments, the first active agent may be the voltage-gated potassium channel (KCNB2) inhibitor and the second active agent may be other active agent, such as the adrenergic receptor α2B (ADRA2B) antagonist.

The dosage ratio of the first active agent and the second active agent may be in the range of 1:100-100:1, 1:80-80:1, 1:50-50:1, 1:40-40:1, 1:30-30:1, 1:20-20:1, 1:10-10:1, 1:5-5:1, 1:4-4:1, 1:3-3:1, 1:2-2:1 or 1:1 by molar ratio, for example, in the single dosage form.

The amount of the first active agent in a unit dosage form may be 1-1000 mg, such as 1, 2, 3, 4, 5, 6, 7, 8, 9, 10, 11, 12, 13, 14, 15, 16, 17, 18, 19, 20, 25, 30, 35, 40, 50, 60, 70, 75, 80, 90, 100, 110, 120, 125, 130, 140, 150, 160, 170, 175, 180, 190, 200, 250, 300, 350, 400, 450, 500, 600, 700, 750, 800, 900, 1000 mg or any range between any two of the above specific values. The amount of the second active agent in a unit dosage form may be 1-1000 mg, such as 1, 2, 3, 4, 5, 6, 7, 8, 9, 10, 11, 12, 13, 14, 15, 16, 17, 18, 19, 20, 25, 30, 35, 40, 50, 60, 70, 75, 80, 90, 100, 110, 120, 125, 130, 140, 150, 160, 170, 175, 180, 190, 200, 250, 300, 350, 400, 450, 500, 600, 700, 750, 800, 900, 1000 mg or any range between any two of the above specific values.

In some embodiments, a combination of three active agents (a first active agent, a second active agent and a third active agent) is used to treat ALS. The first active agent, the second active agent and the third active agent are different from each other. The first active agent, the second active agent and the third active agent are independently selected from the group consisting of a retinoic acid receptor alpha (RARα) agonist, a voltage-gated potassium channel (KCNB2) inhibitor, an adrenergic receptor α2B (ADRA2B) antagonist, a DNA methyltransferase 3 alpha (DNMT3A) antagonist, an insulin like growth factor 1 receptor (IGF1R) inhibitor, a mitogen-activated protein kinase 1 (MAPK1) inhibitor, a nitric oxide synthase 1 (NOS1) inhibitor, a glucocorticoid receptors (NR3C1) antagonist, and a peptidylprolyl Isomerase A (PPIA) antagonist. The two active agents may be formulated in a single dosage form or separately.

In some embodiments, the first active agent may be the retinoic acid receptor alpha (RARα) agonist, the second active agent may be the voltage-gated potassium channel (KCNB2) inhibitor and the third active agent may be the adrenergic receptor α2B (ADRA2B) antagonist.

The dosage ratio of the first active agent, the second active agent and the third active agent may be 1:100:100-100:100:1, 1:80:80-80:80:1, 1:50:50-50:50:1, 1:30:30-30:30:1, 1:20:20-20:20:1, 1:10: 10-10:10:1, 1:5:5-5:5:1, 1:4:4-4:4:1, 1:3:3-3:3:1, 1:2:2-2:2:1 or 1:1:1 by molar ratio, for example, in the single dosage form.

The amount of the first active agent in a unit dosage form may be 1-1000 mg, such as 1, 2, 3, 4, 5, 6, 7, 8, 9, 10, 11, 12, 13, 14, 15, 16, 17, 18, 19, 20, 25, 30, 35, 40, 50, 60, 70, 75, 80, 90, 100, 110, 120, 125, 130, 140, 150, 160, 170, 175, 180, 190, 200, 250, 300, 350, 400, 450, 500, 600, 700, 750, 800, 900, 1000 mg or any range between any two of the above specific values. The amount of the second active agent in a unit dosage form may be 1-1000 mg, such as 1, 2, 3, 4, 5, 6, 7, 8, 9, 10, 11, 12, 13, 14, 15, 16, 17, 18, 19, 20, 25, 30, 35, 40, 50, 60, 70, 75, 80, 90, 100, 110, 120, 125, 130, 140, 150, 160, 170, 175, 180, 190, 200, 250, 300, 350, 400, 450, 500, 600, 700, 750, 800, 900, 1000 mg or any range between any two of the above specific values. The amount of the third active agent in a unit dosage form may be 1-1000 mg, such as 1, 2, 3, 4, 5, 6, 7, 8, 9, 10, 11, 12, 13, 14, 15, 16, 17, 18, 19, 20, 25, 30, 35, 40, 50, 60, 70, 75, 80, 90, 100, 110, 120, 125, 130, 140, 150, 160, 170, 175, 180, 190, 200, 250, 300, 350, 400, 450, 500, 600, 700, 750, 800, 900, 1000 mg or any range between any two of the above specific values.

### Administration

Each of the active agent of the present invention may be administered to the subject via oral, buccal, sublingual, rectal, vaginal, parenteral, intradermal or intranasal or parenteral route. The parenteral administration includes intravenous, intraperitoneal, intradermal, subcutaneous, intramuscular, intracranial, intrathecal, intratumoral, transdermal, transmucosal intraarticular, intra-synovial, intrasternal, intrathecal, intrahepatic, intralesional or intracranial injection or infusion.

The active agent(s) as used herein may be formulated for administration in a pharmaceutical composition in accordance with known techniques. See, for example, Remington, The Science and Practice of Pharmacy (9th Ed. 1995). In the manufacture of a pharmaceutical composition according to the present invention, the active agent is typically admixed with, inter alia, a pharmaceutical acceptable carrier. The carrier must, of course, be acceptable in the sense of being compatible with any other ingredients in the formulation and must not be deleterious to the patient. The carrier may be a solid or a liquid, or both, and is preferably formulated with the compound as a unit-dose formulation, for example, a tablet, which may contain from 0.01% or 0.5% to 95% or 99% by weight of the active agent. One or more active agents may be incorporated in the formulations of the invention, which may be prepared by any of the well-known techniques of pharmacy comprising admixing the components, optionally including one or more accessory ingredients and/or excipients. In some embodiments, any of the compositions, carriers, accessory ingredients excipients and/or the formulations of the invention comprise ingredients that are from either natural or non-natural sources. In other embodiments, any component of the compositions, carriers, accessory ingredients, excipients and/or the formulations of the invention may be provided in a sterile form. Non-limiting examples of a sterile carrier include endotoxin-free water or pyrogen-free water.

In some embodiments, the pharmaceutical composition of the invention is provided as part of a sterile composition/formulation comprising an active agent of the invention and a pharmaceutically acceptable carrier and/or excipient.

Dosage forms suitable for the oral administration include tablet, capsule, powder, pill, granule, suspension, solution or preconcentrate of solution, emulsion or preconcentrates of emulsion. Pharmaceutical acceptable carriers that can be used in an oral dosage form include water, glycols, oils, alcohols, flavoring agents, preservatives, coloring agents and the like. Carriers such as starches, sugars, microcrystalline cellulose, diluents, filler, glidants, granulating agents, lubricants, binders, stabilizers, disintegrating agents and the like can be used to prepare an oral solid preparation such as powder, capsule or tablet.

The diluent includes, but not limited to, microcrystalline cellulose, mannitol, powdered sugar, compressible sugar, dextran, dextrin, spinose, lactose, cellulose powder, sorbitol, sucrose and Talc powder or a combination thereof. The diluent may be 5% to 90% based on the total weight of the oral composition, preferably 10% to 80%, 20%-70%, 30%-60%, 40%-50%.

The disintegrating agent includes, but not limited to, cellulose, alginate, gum, cross-linked polymer, such as cross-linked polyvinylpyrrolidone or crospovidone, croscarmellose sodium, croscarmellose calcium, soybean polysaccharide, sodium starch glycolate, guar gum or any combination thereof. The disintegrating agent may be present in an amount of about 1% to 15%, preferably 2% to 10%, based on the total weight of the oral composition.

The binder includes, but not limited to, starch, cellulose or derivatives thereof, such as microcrystalline cellulose, hydroxypropyl cellulose, hydroxyethyl cellulose and hydroxypropyl methyl cellulose, sucrose, dextrose, corn syrup, polysaccharide, gelatin or any combination thereof. The binder may be present in an amount of 0.01 to 10%, preferably 1% to 10%, based on the total weight of the composition.

The glidant includes, but not limited to, colloidal silicon dioxide, magnesium trisilicate, cellulose powder, talc powder or a combination thereof can be selected. The glidant may be present in an amount of 0.1% to 10%, preferably 0.1% to 0.5%, based on the total weight of the composition.

Dosage forms can be in the form, e.g., of tablets or capsules, and the effective dose may be provided in one or more tablets, capsules or the like, and be provided once a day or throughout the day at intervals, e.g., of 4, 8 or 12 hours. Tablets or capsules, for example, could contain, e.g., 10, 25, 50, 75, 100, 150, 200, 250, 300, 350, 400, 450, 500, 600, 700, 800, 900, 1,000, 1,100, or 1,250 mg of the active agent. For example, administration to a human subject of the active agent of the present invention may comprise a daily dosage in the range of 100-1,250, 150-1,000, 200-800, or 250-750 mg, which daily dosage can be administered either once a day in its entirety or fractions of which are administered throughout the day in intervals. Liquid formulations can also be prepared so that any dosage may readily and conveniently be dispensed.

Parenteral dosage forms are preferably sterile or capable of being sterilized prior to administration to a subject. Examples of parenteral dosage forms include, but are not limited to, solutions ready for injection, dry products ready to be dissolved or suspended in a pharmaceutically acceptable carrier for injection, suspensions ready for injection, and emulsions.

Some suitable carriers that can be used to provide parenteral dosage forms provided herein include, but are not limited to: water for injection; aqueous vehicles such as, but not limited to, sodium chloride injection, Ringer's injection, dextrose injection; water-miscible carriers such as, but not limited to, ethyl alcohol, polyethylene glycol, and polypropylene glycol; and non-aqueous carriers such as, but not limited to, corn oil, cottonseed oil, peanut oil, sesame oil, ethyl oleate, isopropyl myristate, and benzyl benzoate.

Compounds that increase the solubility of one or more of the active agents disclosed herein can also be incorporated into the parenteral dosage forms provided herein. For example, cyclodextrin and its derivatives can be used to increase the solubility of an active agent of the present invention.

It should be understood that a therapeutically effective dose may be determined by a physician, according to such as the type, stage and/or severity of the disease, the condition, age, body weight, sex and response of the subject to be treated, as well as the route of administration.

A therapeutically effective amount is an amount such that when administered to a subject is sufficient to achieve a plasma concentration of from about 0.01 μg/ml to about 100 μg/ml, from about 0.1 μg/ml to about 10 μg/ml, from about 1 μg/ml to about 5 μg/ml.

When administering the active agent of the present invention to a subject, the therapeutically effective amount of each of the active agents generally may be in the range of about 0.5 to about 250 mg/kg, about 1 to about 250 mg/kg, about 2 to about 200 mg/kg, about 3 to about 120 mg/kg, about 5 to about 250 mg/kg, about 10 to about 200 mg/kg, or about 20 to about 120 mg/kg for each active agent of the present invention. In some embodiments, the therapeutically effective amount may be 0.5 mg/kg, 1 mg/kg, 2 mg/kg, 3 mg/kg, 4 mg/kg, 5 mg/kg, 6 mg/kg, 8 mg/kg, 10 mg/kg, 20 mg/kg, 25 mg/kg, 40 mg/kg, 50 mg/kg, 60 mg/kg, 75 mg/kg, 100 mg/kg, 120 mg/kg, 150 mg/kg, 175 mg/kg, 200 mg/kg, 225 mg/kg, 250 mg/kg or 300 mg/kg.

Each of the active agents of the present invention may be administered once or twice one day; or once every 2, 3, 4, 5, 6, 7, 8, 9 or 10 days or once every 1, 2 or 3 weeks. In some embodiments, each of the active agents of the present invention may be administered in a five times weekly scheme. In the five times weekly scheme, the administration may be done on five consecutive days (once daily) followed by two consecutive days off.

The term “kit”, as used herein, refers to a package and, as a rule, instruction for use. An active agent or a pharmaceutical composition in a kit can be in any of a variety of forms suitable for distribution in a kit. Such forms can include a liquid, powder, tablet, suspension and the like. Two or more active agents may be provided in separate containers suitable for administration separately, or alternatively may be provided in a composition in a single container in the package. The kit may contain an amount sufficient for one or more dosages of agents according to the treatment methods. The instruction for use generally comprises a literal statement of how to treat a disease (such as ALS) with the agents in the kit.

It should be understood that the combination or pharmaceutical composition of the present invention may include other therapeutic agents or therapies, such as biological therapeutic agents and/or chemotherapeutic agents in addition to the active agents of the present invention. The method may include administration of other therapeutic agents or therapies, such as biological therapeutic agents and/or chemotherapeutic agents in addition to administration of the active agents of the present invention. Other therapeutic agents or therapies may be administered simultaneously, separately or sequentially with the therapeutic agents of the present invention.

### EXAMPLES

### Example 1 Identification of Therapeutic Targets and Drug Repurposing Candidates for Amyotrophic Lateral Sclerosis Using PandaOmics™—an AI-Enabled Biological Target Discovery Platform

### 1. Abstract

Amyotrophic lateral sclerosis (ALS) is a severe neurodegenerative disease that results in progressive loss of motor neurons. After decades of research, the pathogenic mechanisms underlying ALS remain ill defined, and there is no effective treatment for the disease. This calls for an urgent need for a new therapeutic regimen. Herein, we applied PandaOmics™, an AI-driven target discovery platform, to analyze the expression profiles of central nervous system (CNS) samples (237 cases; 91 controls) from public datasets, and iPSC-derived motor neurons (diMN) samples (135 cases; 31 controls) from Answer ALS project. Several well-characterized mechanisms in ALS pathology are found to be dysregulated, including the immune system, RNA metabolism, excitotoxicity, as well as programmed cell death. Twenty-three and twelve potential therapeutic targets with multiple levels of novelty are identified from CNS and diMN samples, respectively. Targets identified from CNS data mainly contribute to neuronal cell death and inflammation, which are recognized as late-stage signatures of ALS. In contrast, more diMN targets are attributed to the early-stage signatures. Combining the usage of diMN and post-mortem CNS samples could provide an in-depth understanding of ALS disease progression. To accelerate novel target discovery and drug investigation for ALS, we established the platform ALS.AI (http://als.ai/) for the release of novel therapeutic targets and curated drug repurposing candidates. By integrating all information together, our study could facilitate the identification of new drugs or drug combos for ALS patients and open a new path to search for the cure of other human diseases.

### 2. Introduction

Thanks to the advances in genomic profiling techniques, numerous genome-wide association studies were conducted to screen for common genetic variants in ALS and have identified novel candidates as genetic predisposition or biomarkers (e.g. ACSL5, KIF5A, ATXN2, and MOBP). Furthermore, the utility of both cellular and animal models with ALS-linked gene variants helps determine the potential interacting partners of those ALS-linked genes, providing multiple lines of evidence for uncovering disease pathology[4]. Here, we applied PandaOmics™—an artificial intelligence (AI)-powered target discovery platform—to explore dysregulated genes and altered pathways across various ALS-related datasets. To enrich the diversity of data sources, we utilized post-mortem central nervous system (CNS) tissues and induced pluripotent stem cell (iPSC)-differentiated motor neurons derived from ALS patients to perform target discovery. By customizing the filter setting, 21 high-confidence and 14 novel candidates were selected as promising ALS therapeutic targets. Proposed targets will be released onto the platform ALS.AI (http://als.ai/). The aim of this study is to demonstrate the utilization of the AI-driven target discovery platform—PandaOmics™—to find therapeutic targets for ALS.

### 3. Abbreviations

AD: Alzheimer's disease;
AI: Artificial intelligence;
ALS: Amyotrophic lateral sclerosis;
fALS: Familial ALS; sALS: Sporadic ALS;
CNS: Central nervous system;
Cn: Calcineurin;
diMN: Direct iPSC-derived motor neuron;
ER: Endoplasmic reticulum;
iPSC: Induced pluripotent stem cell;
KOL: Key opinion leader;
LFC: log2-transformed fold change;
MN: Motor neuron;
NADPH: Nicotinamide adenine dinucleotide phosphate;
NO: Nitric oxide;
nNOS: Neuronal nitric oxide synthase;
PD: Parkinson's disease;
UPR: unfolded protein response.

### 4. Methods

### Data Sources and Availability

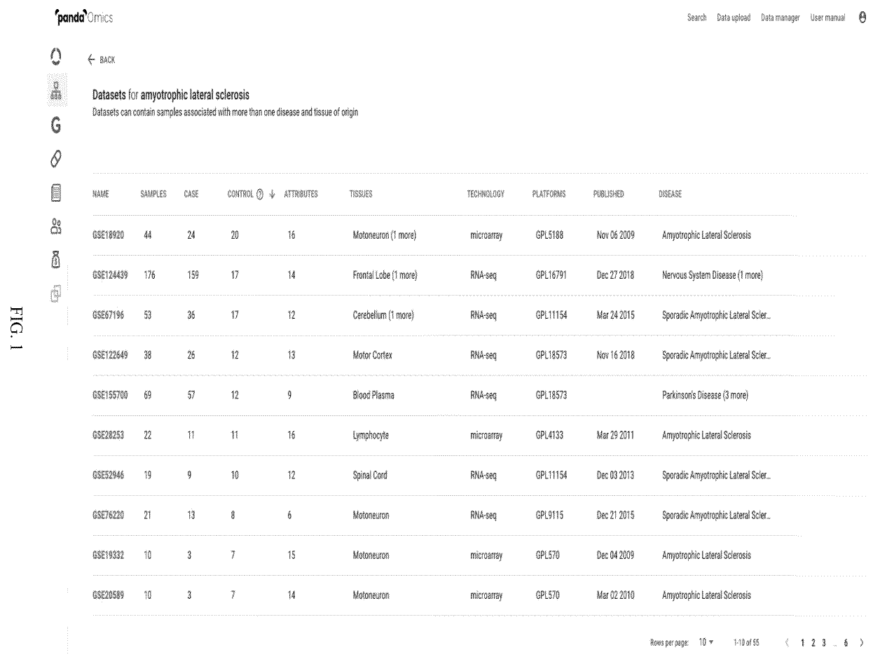

Microarray and RNA-seq datasets for ALS patients and control samples were retrieved from public repositories and processed by PandaOmics™ for downstream analysis and target identification. Over fifty ALS-related datasets of various tissue sources are available in PandaOmics™ (FIG. 1), including datasets of post-mortem CNS tissues, iPSC-derived neurons, blood, etc. For each dataset, samples can be divided into subgroups based on their clinical subtypes or other phenotypic attributes. In addition, transcriptomics and proteomics data of the direct iPSC-derived motor neurons (diMNs), generated from ALS patients and neurologically healthy subjects in the Answer ALS project[5] were uploaded to PandaOmics™ and incorporated in our analyses. The diMNs are differentiated from the primary peripheral blood mononuclear cell-derived induced pluripotent cell lines. Detailed protocol for the diMN generation was described by Rothstein et al. (2020)[5].

The raw transcriptomics data of CNS comparisons were available in public repositories that could be retrieved by their series identifiers. In addition, transcriptomics and proteomics profiles of the diMN samples were available to investigators upon request and approval from the Answer ALS.

### Dataset and Comparison Selection

Given that the degeneration of motor neurons in the brain and spinal cord underlie ALS pathogenesis, CNS tissue datasets were selected for analysis in the present study. Samples carrying one of the four major fALS-linked gene variations (SOD1, TDP-43, FUS, and C9orf72) were classified as the fALS group, and those with unspecified gene variations as the sALS group, yielding five familial as well as seven sporadic ALS case-control comparisons (Table 1).

TABLE 1
ALS case-control comparisons using CNS samples

| Data series | Platform | Technology | Source | Mutant gene | # Case | # Control | Year |
|---|---|---|---|---|---|---|---|
| Familial | | | | | | | |
| ALS | | | | | | | |
| E−MTAB- | A-MEXP- | Microarray | Motor Cortex | C9orf72 | 3 | 3 | 2013 |
| 1925 | 2246 | | | | | | |
| GSE67196 | GPL11154 | RNA-seq | Cerebellum | C9orf72 | 8 | 8 | 2015 |
| GSE67196 | GPL11154 | RNA-seq | Frontal Cortex | C9orf72 | 8 | 9 | 2015 |
| GSE68605 | GPL570 | Microarray | Motor Neurons | C9orf72 | 8 | 3 | 2015 |
| GSE20589 | GPL570 | Microarray | Motor Neurons | SOD1 | 3 | 7 | 2010 |
| Sporadic | | | | | | | |
| ALS | | | | | | | |
| GSE122649 | GPL18573 | RNA-seq | Motor Cortex | — | 26 | 12 | 2018 |
| GSE124439 | GPL16791 | RNA-seq | Frontal Cortex | — | 65 | 9 | 2018 |
| GSE124439 | GPL16791 | RNA-seq | Motor Cortex | — | 80 | 8 | 2018 |
| GSE19332 | GPL570 | Microarray | CNS tissues | — | 3 | 7 | 2009 |
| GSE76220 | GPL9115 | RNA-seq | Spinal Motor | — | 13 | 8 | 2015 |
| | | | Neurons | | | | |
| GSE67196 | GPL11154 | RNA-seq | Cerebellum | — | 10 | 8 | 2015 |
| GSE67196 | GPL11154 | RNA-seq | Frontal Cortex | — | 10 | 9 | 2015 |

The non-Hispanic and non-Latino whites represent the largest ethnic group in the datasets from Answer ALS amounting to over 85% of the total samples. In this regard, diMN samples belonging to this ethnic group with both transcriptomics and proteomics data were selected for the current analysis. The samples were further divided into 25 fALS and 110 sALS based on the presence or absence of the family history of ALS occurrence. As a result, two subtype-dependent comparisons were built using the diMN transcriptomics and proteomics data respectively (Table 2).

TABLE 2
ALS case-control comparisons using diMN samples

| | | | # | # | # Detected |
|---|---|---|---|---|---|
| ALS subtype | Technology | Source | Case | Control | genes |
| Transcriptomics | | | | | |
| fALS | RNA-seq | direct iPSC- | 25 | 31 | 37,073 |
| | | derived motor | | | |
| | | neuron | | | |
| sALS | RNA-seq | direct iPSC- | 110 | 31 | 37,073 |
| | | derived motor | | | |
| | | neuron | | | |
| Proteomics | | | | | |
| fALS | SWATH- | direct iPSC- | 25 | 31 | 4,468 |
| | MS | derived motor | | | |
| | | neuron | | | |
| sALS | SWATH- | direct iPSC- | 110 | 31 | 4,468 |
| | MS | derived motor | | | |
| | | neuron | | | |

### Pathway Analysis

The degree of pathway dysregulation was determined by the PandaOmics™ proprietary iPANDA algorithm accounting for the differential gene expression and the topological decomposition of pathways[6]. We analyzed both CNS and diMN comparison groups for pathway dysregulation based on Version 73 of the Reactome database. For each group, a pathway was considered as dysregulated when 1) its alteration was unidirectional in greater than or equal to 80% of all the comparisons of the ALS subtype, and 2) the |iPANDA| value reached the threshold of 0.01 in at least one comparison of a subtype. Networks of dysregulated pathways were constructed using EnrichmentMap in Cytoscape. The hierarchical level of pathways retrieved from the Reactome database was employed as the basis for the annotation of pathway clusters in the networks.

### Filter Setting for Target Identification

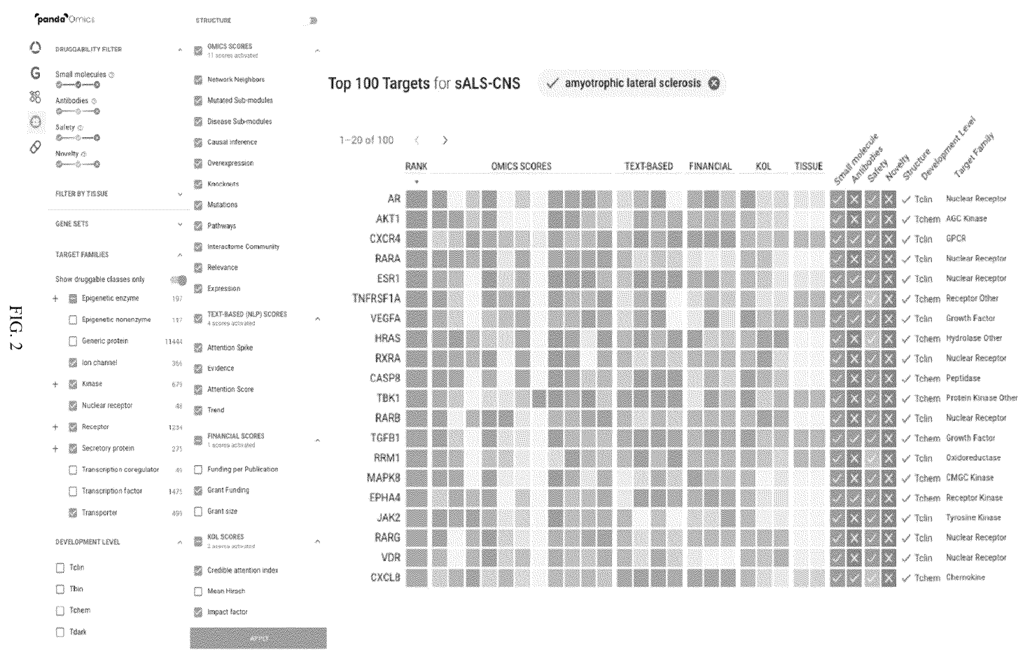

To identify potential targets for ALS, meta-analyses were performed on the five CNS fALS comparisons, seven CNS sALS comparisons, diMN transcriptomics, and proteomics comparisons independently. PandaOmics™ ranks related genes and identifies potential targets with AI hypothesis generation models based on 21 scores from Omics, Text-based, Financial, and Key opinion leader (KOL) categories. In addition, Druggability filters (small molecules, antibodies, safety, novelty), Tissue specificity filters, Target family filters, and Development filters could be applied to further refine the list to meet the user's research goals. For high confidence targets identification, we customized the Druggability filters to screen targets already associated with small molecules and have a median safety level (FIG. 2). Simultaneously, Druggable classes, Omics scores, Text-based scores, Grant Funding, as well as KOL scores (credible attention index and impact factor) were applied. A list of high-confidence druggable targets was ranked in descending order based on their metascores, and the top-50 targets were selected for further investigation.

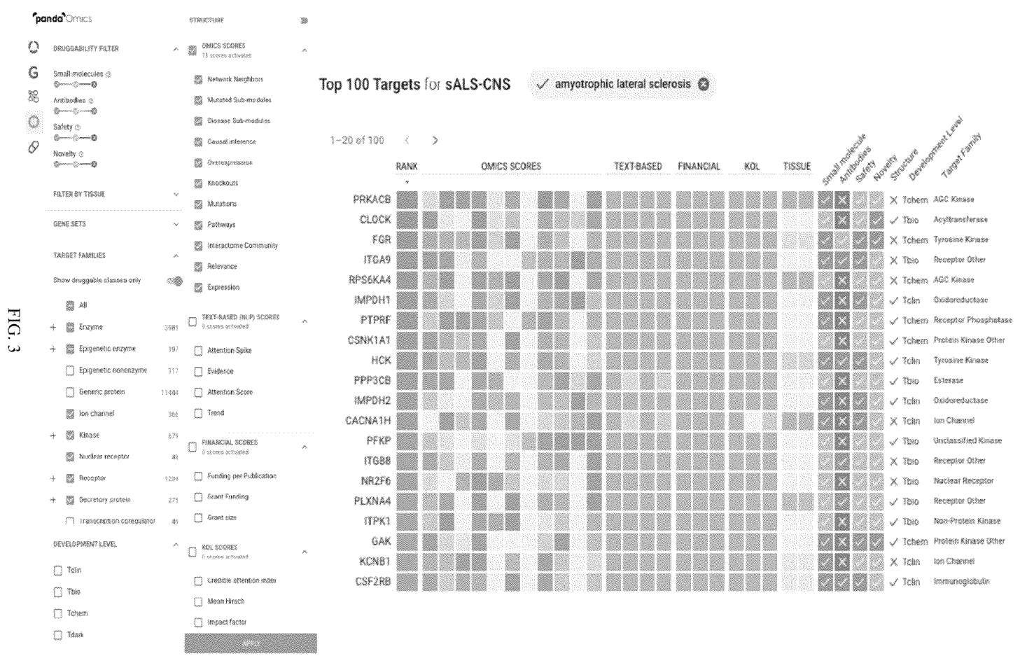

Similarly, novel ALS targets could be identified without prior knowledge by restricting the Druggability filter to the high novelty level, selecting only the Omics scores, and disabling the Text-based, Financial, and KOL scores (FIG. 3). After recalculating the metascores with the new criteria, the top-50 ranked genes were selected as novel targets for further analysis.

### Validation of the Scoring Approach

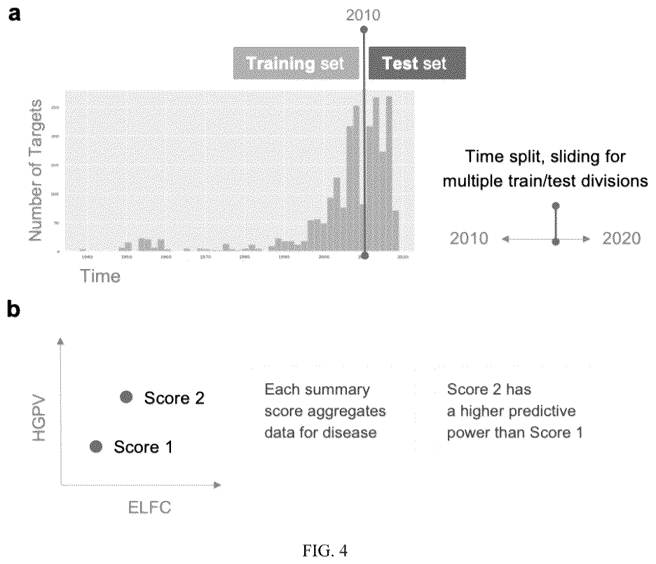

The “time machine” approach was applied for the validation of the ability of a model to identify the truly novel targets of the disease of interest. The data before a given year was used as training data and the trained model was then evaluated based on the targets entering the clinical phase after the given year (FIG. 4a). Two validation metrics were used to validate the scoring approach. ELFC refers to the log fold change of enrichment shows how much the top of the list is enriched by known targets and is calculated by the formula (I):

$$ELFC ( score ) = {log}_{2} ( \frac{{targets}_{k} \cdot N}{k \cdot {targets}_{N}} ) \qquad ( I )$$

where targetsₖ is the number of known targets for this disease in top-k (or 0.1 if there are none), and targets_{N} is the total number of known targets for this disease among the genes that are available for a particular PandaOmics score. And HGPV stands for the statistical significance of the effect and shows how likely the same level of enrichment can be achieved from the random distribution and is calculated by the formula (II):

$$HGPV ( score ) = - {log}_{10} ( 1 - hgcdf ( {targets}_{k} , k , {targets}_{N} , N ) ) \qquad ( II )$$

where hgcdf is a hypergeometric cumulative distribution function. A score with higher values of ELFC and HGPV corresponds to the higher predictive power of the target-disease association (FIG. 4b).

### 5. Results

### Clustering of Dysregulated Pathways

The Reactome database has provided the hierarchical organization of pathways that related pathways are grouped into broader domains of biological functions[7]. Therefore, all the pathways analyzed here could be classified into 27 biological processes, each of which corresponds to one top-level pathway according to the Reactome hierarchy. In CNS groups, dysregulated pathways in ALS patients are overrepresented in the immune system process (fALS, adjusted p=3.26E-7), signal transduction process (sALS, adjusted p=9.2E-5), and hemostasis (fALS, adjusted p=0.0054). The diMN transcriptomics groups were enriched with dysregulated pathways belonging to the digestion and absorption process (fALS, adjusted p=0.0059), and the process of protein metabolism (sALS, adjusted p=0.0093). The dysregulated pathways in the diMN proteomics groups were overrepresented in the processes of disease (sALS, adjusted p=3.38E-8), DNA repair (fALS, adjusted p=0.0233), and developmental biology (fALS, adjusted p=0.0255). The details of dysregulated pathways in different biological processes are available in Table 3.

TABLE 3
Enrichment of dysregulated pathways in different biological processes.

| | | | # | | | |
|---|---|---|---|---|---|---|
| | | | Dysregulated | | pvalue | adjusted p |
| Main biological | | ALS | pathways | Fold | (hypergeometric | (Bonferroni |
| process | Tissue | subtype | in process | enrichment | test) | correction) |
| Immune System | CNS | fALS | 41 | 1.4200 | 1.02E−08 | 3.26E−07 |
| Signal | CNS | sALS | 40 | 0.9629 | 2.87E−06 | 9.20E−05 |
| Transduction | | | | | | |
| Hemostasis | CNS | fALS | 11 | 2.4394 | 1.68E−04 | 5.39E−03 |
| Gene expression | CNS | sALS | 14 | 1.1109 | 4.96E−03 | 1.59E−01 |
| (Transcription) | | | | | | |
| Metabolism of | CNS | sALS | 7 | 1.7847 | 1.09E−02 | 3.48E−01 |
| RNA | | | | | | |
| Extracellular | CNS | fALS | 5 | 2.4026 | 1.19E−02 | 3.81E−01 |
| matrix | | | | | | |
| organization | | | | | | |
| Cell-Cell | CNS | fALS | 4 | 2.8563 | 1.53E−02 | 4.91E−01 |
| communication | | | | | | |
| Programmed Cell | CNS | fALS | 8 | 1.3138 | 1.83E−02 | 5.85E−01 |
| Death | | | | | | |
| Cell Cycle | CNS | fALS | 15 | 0.4224 | 9.76E−02 | 1.00E+00 |
| Protein | CNS | sALS | 1 | 2.7394 | 2.41E−01 | 1.00E+00 |
| localization | | | | | | |
| Organelle | CNS | fALS | 2 | 0.9281 | 2.78E−01 | 1.00E+00 |
| biogenesis and | | | | | | |
| maintenance | | | | | | |
| Muscle | CNS | sALS | 1 | 1.3371 | 3.56E−01 | 1.00E+00 |
| contraction | | | | | | |
| Organelle | CNS | sALS | 1 | 0.5581 | 4.84E−01 | 1.00E+00 |
| biogenesis and | | | | | | |
| maintenance | | | | | | |
| Disease | CNS | fALS | 15 | −0.0413 | 6.14E−01 | 1.00E+00 |
| Programmed Cell | CNS | sALS | 2 | −0.0652 | 6.41E−01 | 1.00E+00 |
| Death | | | | | | |
| Cellular responses | CNS | sALS | 1 | −0.1871 | 7.20E−01 | 1.00E+00 |
| to external stimuli | | | | | | |
| Gene expression | CNS | fALS | 9 | −0.1603 | 7.63E−01 | 1.00E+00 |
| (Transcription) | | | | | | |
| Disease | CNS | sALS | 8 | −0.1736 | 7.70E−01 | 1.00E+00 |
| Transport of | CNS | sALS | 2 | −0.3075 | 7.96E−01 | 1.00E+00 |
| small molecules | | | | | | |
| DNA Repair | CNS | sALS | 2 | −0.3662 | 8.35E−01 | 1.00E+00 |
| Vesicle-mediated | CNS | fALS | 2 | −0.3747 | 8.44E−01 | 1.00E+00 |
| transport | | | | | | |
| Metabolism of | CNS | fALS | 7 | −0.2638 | 8.56E−01 | 1.00E+00 |
| proteins | | | | | | |
| Metabolism | CNS | sALS | 9 | −0.2488 | 8.67E−01 | 1.00E+00 |
| Cellular responses | CNS | fALS | 1 | −0.4970 | 8.77E−01 | 1.00E+00 |
| to external stimuli | | | | | | |
| Metabolism of | CNS | sALS | 3 | −0.4901 | 9.43E−01 | 1.00E+00 |
| proteins | | | | | | |
| Transport of | CNS | fALS | 2 | −0.5715 | 9.56E−01 | 1.00E+00 |
| small molecules | | | | | | |
| Signal | CNS | fALS | 25 | −0.2409 | 9.61E−01 | 1.00E+00 |
| Transduction | | | | | | |
| Immune System | CNS | sALS | 6 | −0.4276 | 9.62E−01 | 1.00E+00 |
| Developmental | CNS | sALS | 1 | −0.6884 | 9.65E−01 | 1.00E+00 |
| Biology | | | | | | |
| Metabolism | CNS | fALS | 12 | −0.3802 | 9.82E−01 | 1.00E+00 |
| Developmental | CNS | fALS | 1 | −0.8072 | 9.96E−01 | 1.00E+00 |
| Biology | | | | | | |
| Cell Cycle | CNS | sALS | 1 | −0.8467 | 9.99E−01 | 1.00E+00 |
| Digestion and | diMN | fALS | 2 | 55.9538 | 4.50E−04 | 5.85E−03 |
| absorption | transcriptomics | | | | | |
| Metabolism of | diMN | sALS | 7 | 3.3626 | 7.13E−04 | 9.27E−03 |
| proteins | transcriptomics | | | | | |
| Immune System | diMN | fALS | 5 | 2.6323 | 8.00E−03 | 1.04E−01 |
| | transcriptomics | | | | | |
| Metabolism of | diMN | sALS | 3 | 3.3759 | 2.93E−02 | 3.81E−01 |
| RNA | transcriptomics | | | | | |
| Transport of | diMN | sALS | 3 | 2.8086 | 4.19E−02 | 5.44E−01 |
| small molecules | transcriptomics | | | | | |
| Metabolism | diMN | fALS | 4 | 1.5426 | 6.18E−02 | 8.03E−01 |
| | transcriptomics | | | | | |
| Neuronal System | diMN | sALS | 2 | 1.5870 | 1.80E−01 | 1.00E+00 |
| | transcriptomics | | | | | |
| Disease | diMN | sALS | 4 | 0.5150 | 2.68E−01 | 1.00E+00 |
| | transcriptomics | | | | | |
| Immune System | diMN | sALS | 3 | 0.0493 | 5.57E−01 | 1.00E+00 |
| | transcriptomics | | | | | |
| Developmental | diMN | sALS | 1 | 0.1426 | 5.92E−01 | 1.00E+00 |
| Biology | transcriptomics | | | | | |
| Signal | diMN | fALS | 2 | −0.2526 | 7.83E−01 | 1.00E+00 |
| Transduction | transcriptomics | | | | | |
| Signal | diMN | sALS | 3 | −0.4602 | 9.39E−01 | 1.00E+00 |
| Transduction | transcriptomics | | | | | |
| Metabolism | diMN | sALS | 1 | −0.6939 | 9.70E−01 | 1.00E+00 |
| | transcriptomics | | | | | |
| Disease | diMN | sALS | 14 | 5.2248 | 1.69E−09 | 3.38E−08 |
| | proteomics | | | | | |
| DNA Repair | diMN | fALS | 6 | 3.8266 | 1.17E−03 | 2.33E−02 |
| | proteomics | | | | | |
| Developmental | diMN | fALS | 6 | 3.7462 | 1.28E−03 | 2.55E−02 |
| Biology | proteomics | | | | | |
| Reproduction | diMN | fALS | 2 | 12.5604 | 8.50E−03 | 1.70E−01 |
| | proteomics | | | | | |
| Gene expression | diMN | fALS | 7 | 1.6793 | 1.30E−02 | 2.59E−01 |
| (Transcription) | proteomics | | | | | |
| Neuronal System | diMN | fALS | 4 | 2.5820 | 2.35E−02 | 4.69E−01 |
| | proteomics | | | | | |
| Cell-Cell | diMN | fALS | 2 | 6.9103 | 2.50E−02 | 5.00E−01 |
| communication | proteomics | | | | | |
| Muscle | diMN | sALS | 1 | 9.0598 | 9.54E−02 | 1.00E+00 |
| contraction | proteomics | | | | | |
| Gene expression | diMN | sALS | 3 | 0.9471 | 1.96E−01 | 1.00E+00 |
| (Transcription) | proteomics | | | | | |
| Programmed Cell | diMN | sALS | 1 | 1.0120 | 3.97E−01 | 1.00E+00 |
| Death | proteomics | | | | | |
| Transport of | diMN | sALS | 1 | 0.4903 | 4.96E−01 | 1.00E+00 |
| small molecules | proteomics | | | | | |
| Developmental | diMN | sALS | 1 | 0.3413 | 5.34E−01 | 1.00E+00 |
| Biology | proteomics | | | | | |
| Metabolism of | diMN | fALS | 2 | −0.1371 | 6.86E−01 | 1.00E+00 |
| proteins | proteomics | | | | | |
| Transport of | diMN | fALS | 1 | −0.1211 | 6.89E−01 | 1.00E+00 |
| small molecules | proteomics | | | | | |
| Cell Cycle | diMN | fALS | 2 | −0.2219 | 7.41E−01 | 1.00E+00 |
| | proteomics | | | | | |
| Metabolism of | diMN | sALS | 1 | −0.2684 | 7.58E−01 | 1.00E+00 |
| proteins | proteomics | | | | | |
| Disease | diMN | fALS | 2 | −0.4756 | 9.08E−01 | 1.00E+00 |
| | proteomics | | | | | |
| Signal | diMN | fALS | 4 | −0.5017 | 9.74E−01 | 1.00E+00 |
| Transduction | proteomics | | | | | |
| Metabolism | diMN | fALS | 1 | −0.7881 | 9.94E−01 | 1.00E+00 |
| | proteomics | | | | | |
| Signal | diMN | sALS | 1 | −0.7888 | 9.95E−01 | 1.00E+00 |
| Transduction | proteomics | | | | | |

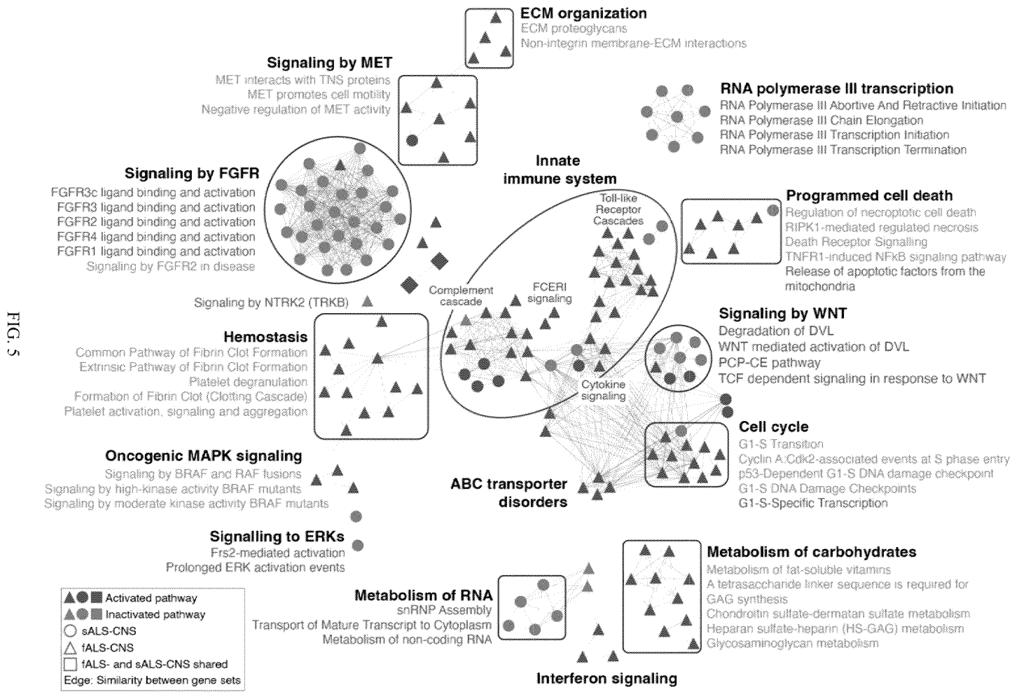

Furthermore, pathways with similar gene contents can be connected to form clusters. As shown in FIG. 5, the most prominent cluster of the dysregulated pathways in CNS ALS groups relative to healthy groups was associated with activated innate immune system, which consisted of activated pathways of the Toll-like receptor cascades, FCERI signaling, cytokine signaling, and regulation of complement cascade. Moreover, pathways of programmed cell death, and cell cycle pathways associated with G1-S DNA damage checkpoint were activated. Other activated clusters include pathways of the extracellular matrix organization, MET signaling, hemostasis, oncogenic MAPK signaling, ABC transporter disorders, interferon signaling, and carbohydrate metabolism. On the contrary, pathways of FGFR signaling, RNA metabolism, and RNA polymerase III transcription were inhibited. In addition, pathways of RNA polymerase I and II transcription, mitochondrial protein import, and NCAM signaling for neurite out-growth were inhibited as well (Table 4). Notably, there were only a few dysregulated pathways overlapping between fALS and sALS groups (i.e. upregulated pathway of erythropoietin activates PI3-kinase annotated as square in FIG. 5), and most clusters were specific to a sole ALS subtype. For example, the clusters of FGFR signaling, RNA metabolism, and RNA polymerase III transcription mainly contained inhibited pathways identified in the sALS but not the fALS groups.

TABLE 4
Dysregulated pathways in CNS, diMN transcriptomics and diMN proteomics groups.
Dysregulated pathways in CNS comparisons

| Main | | | | | | |
|---|---|---|---|---|---|---|
| biological | | fALS | fALS | sALS | sALS | |
| process | Pathway | (% iPanda > 0) | (% iPanda < 0) | (% iPanda > 0) | (% iPanda < 0) | Direction |
| Cell Cycle | APC-C:Cdc20 | 20 | 80 | 28.57 | 71.43 | fALS |
| | mediated | | | | | Down |
| | degradation of | | | | | |
| | Cyclin B | | | | | |
| Cell Cycle | Inactivation of | 20 | 80 | 28.57 | 71.43 | fALS |
| | APC-C via | | | | | Down |
| | direct inhibition | | | | | |
| | of the APC-C | | | | | |
| | complex | | | | | |
| Cell Cycle | Inhibition of the | 20 | 80 | 28.57 | 71.43 | fALS |
| | proteolytic | | | | | Down |
| | activity of APC- | | | | | |
| | C required for | | | | | |
| | the onset of | | | | | |
| | anaphase by | | | | | |
| | mitotic spindle | | | | | |
| | checkpoint | | | | | |
| | components | | | | | |
| Cell Cycle | Chk1- | 80 | 20 | 42.86 | 14.29 | fALS Up |
| | Chk2(Cds1) | | | | | |
| | mediated | | | | | |
| | inactivation of | | | | | |
| | Cyclin B:Cdk1 | | | | | |
| | complex | | | | | |
| Cell Cycle | Condensation of | 80 | 0 | 57.14 | 42.86 | fALS Up |
| | Prometaphase | | | | | |
| | Chromosomes | | | | | |
| Cell Cycle | Cyclin A:Cdk2- | 80 | 20 | 57.14 | 42.86 | fALS Up |
| | associated | | | | | |
| | events at S phase | | | | | |
| | entry | | | | | |
| Cell Cycle | Cyclin E | 80 | 20 | 42.86 | 57.14 | fALS Up |
| | associated | | | | | |
| | events during | | | | | |
| | G1-S transition | | | | | |
| Cell Cycle | G1-S DNA | 80 | 20 | 57.14 | 42.86 | fALS Up |
| | Damage | | | | | |
| | Checkpoints | | | | | |
| Cell Cycle | G1-S Transition | 80 | 20 | 57.14 | 42.86 | fALS Up |
| Cell Cycle | Mitotic G1 | 80 | 20 | 42.86 | 57.14 | fALS Up |
| | phase and G1-S | | | | | |
| | transition | | | | | |
| Cell Cycle | p53-Dependent | 80 | 20 | 57.14 | 42.86 | fALS Up |
| | G1 DNA | | | | | |
| | Damage | | | | | |
| | Response | | | | | |
| Cell Cycle | p53-Dependent | 80 | 20 | 57.14 | 42.86 | fALS Up |
| | G1-S DNA | | | | | |
| | damage | | | | | |
| | checkpoint | | | | | |
| Cell Cycle | SCF(Skp2)- | 80 | 20 | 57.14 | 42.86 | fALS Up |
| | mediated | | | | | |
| | degradation of | | | | | |
| | p27-p21 | | | | | |
| Cell Cycle | Transcriptional | 80 | 0 | 28.57 | 57.14 | fALS Up |
| | activation of cell | | | | | |
| | cycle inhibitor | | | | | |
| | p21 | | | | | |
| Cell Cycle | Transcriptional | 80 | 0 | 28.57 | 57.14 | fALS Up |
| | activation of p53 | | | | | |
| | responsive genes | | | | | |
| Cell Cycle | G1-S-Specific | 40 | 60 | 14.29 | 85.71 | sALS |
| | Transcription | | | | | Down |
| Cell-Cell | Adherens | 80 | 0 | 57.14 | 42.86 | fALS Up |
| communication | junctions | | | | | |
| | interactions | | | | | |
| Cell-Cell | Cell junction | 100 | 0 | 28.57 | 71.43 | fALS Up |
| communication | organization | | | | | |
| Cell-Cell | Cell-cell | 80 | 20 | 28.57 | 71.43 | fALS Up |
| communication | junction | | | | | |
| | organization | | | | | |
| Cell-Cell | Tight junction | 80 | 20 | 28.57 | 71.43 | fALS Up |
| communication | interactions | | | | | |
| Cellular | Response of | 80 | 0 | 28.57 | 42.86 | fALS Up |
| responses to | EIF2AK1 (HRI) | | | | | |
| external | to heme | | | | | |
| stimuli | deficiency | | | | | |
| Cellular | HSP90 | 60 | 40 | 85.71 | 14.29 | sALS Up |
| responses to | chaperone cycle | | | | | |
| external | for steroid | | | | | |
| stimuli | hormone | | | | | |
| | receptors (SHR) | | | | | |
| Developmental | CRMPs in | 0 | 100 | 71.43 | 28.57 | fALS |
| Biology | Sema3A | | | | | Down |
| | signaling | | | | | |
| Developmental | NCAM | 40 | 60 | 14.29 | 85.71 | sALS |
| Biology | signaling for | | | | | Down |
| | neurite out- | | | | | |
| | growth | | | | | |
| Disease | Interactions of | 20 | 80 | 42.86 | 57.14 | fALS |
| | Vpr with host | | | | | Down |
| | cellular proteins | | | | | |
| Disease | Vpr-mediated | 20 | 80 | 57.14 | 42.86 | fALS |
| | nuclear import | | | | | Down |
| | of PICS | | | | | |
| Disease | ABC transporter | 80 | 20 | 71.43 | 28.57 | fALS Up |
| | disorders | | | | | |
| Disease | Defective CFTR | 80 | 20 | 71.43 | 28.57 | fALS Up |
| | causes cystic | | | | | |
| | fibrosis | | | | | |
| Disease | Diseases | 80 | 0 | 42.86 | 14.29 | fALS Up |
| | associated with | | | | | |
| | the TLR | | | | | |
| | signaling | | | | | |
| | cascade | | | | | |
| Disease | Diseases of | 80 | 0 | 42.86 | 14.29 | fALS Up |
| | Immune System | | | | | |
| Disease | Disorders of | 80 | 20 | 71.43 | 28.57 | fALS Up |
| | transmembrane | | | | | |
| | transporters | | | | | |
| Disease | IkBA variant | 80 | 0 | 42.86 | 14.29 | fALS Up |
| | leads to EDA-ID | | | | | |
| Disease | Signaling by | 80 | 20 | 71.43 | 28.57 | fALS Up |
| | BRAF and RAF | | | | | |
| | fusions | | | | | |
| Disease | Signaling by | 80 | 20 | 28.57 | 71.43 | fALS Up |
| | FGFR2 in | | | | | |
| | disease | | | | | |
| Disease | Signaling by | 80 | 20 | 57.14 | 42.86 | fALS Up |
| | high-kinase | | | | | |
| | activity BRAF | | | | | |
| | mutants | | | | | |
| Disease | Signaling by | 80 | 20 | 57.14 | 42.86 | fALS Up |
| | moderate kinase | | | | | |
| | activity BRAF | | | | | |
| | mutants | | | | | |
| Disease | Uptake and | 80 | 20 | 42.86 | 57.14 | fALS Up |
| | actions of | | | | | |
| | bacterial toxins | | | | | |
| Disease | Viral mRNA | 80 | 0 | 71.43 | 14.29 | fALS Up |
| | Translation | | | | | |
| Disease | Infection with | 80 | 0 | 85.71 | 14.29 | fALS Up; |
| | Mycobacterium | | | | | sALS Up |
| | tuberculosis | | | | | |
| Disease | FGFR3 mutant | 60 | 40 | 14.29 | 85.71 | sALS |
| | receptor | | | | | Down |
| | activation | | | | | |
| Disease | Signaling by | 60 | 40 | 14.29 | 85.71 | sALS |
| | activated point | | | | | Down |
| | mutants of | | | | | |
| | FGFR3 | | | | | |
| Disease | FCGR3A- | 100 | 0 | 85.71 | 14.29 | sALS Up |
| | mediated | | | | | |
| | phagocytosis | | | | | |
| Disease | Leishmania | 100 | 0 | 85.71 | 14.29 | sALS Up |
| | phagocytosis | | | | | |
| Disease | Parasite | 100 | 0 | 85.71 | 14.29 | sALS Up |
| | infection | | | | | |
| Disease | Signaling by | 20 | 20 | 85.71 | 0 | sALS Up |
| | WNT in cancer | | | | | |
| Disease | XAV939 | 20 | 0 | 85.71 | 0 | sALS Up |
| | inhibits | | | | | |
| | tankyrase, | | | | | |
| | stabilizing | | | | | |
| | AXIN | | | | | |
| DNA Repair | Displacement of | 0 | 100 | 0 | 85.71 | sALS |
| | DNA | | | | | Down |
| | glycosylase by | | | | | |
| | APEX1 | | | | | |
| DNA Repair | Fanconi Anemia | 40 | 60 | 14.29 | 85.71 | sALS |
| | Pathway | | | | | Down |
| Extracellular | ECM | 80 | 20 | 71.43 | 28.57 | fALS Up |
| matrix | proteoglycans | | | | | |
| organization | | | | | | |
| Extracellular | Integrin cell | 80 | 20 | 57.14 | 42.86 | fALS Up |
| matrix | surface | | | | | |
| organization | interactions | | | | | |
| Extracellular | Laminin | 80 | 20 | 42.86 | 57.14 | fALS Up |
| matrix | interactions | | | | | |
| organization | | | | | | |
| Extracellular | Non-integrin | 80 | 20 | 57.14 | 42.86 | fALS Up |
| matrix | membrane-ECM | | | | | |
| organization | interactions | | | | | |
| Extracellular | Syndecan | 80 | 0 | 71.43 | 28.57 | fALS Up |
| matrix | interactions | | | | | |
| organization | | | | | | |
| Gene | Regulation of | 80 | 20 | 28.57 | 71.43 | fALS Up |
| expression | MECP2 | | | | | |
| (Transcription) | expression and | | | | | |
| | activity | | | | | |
| Gene | RUNX1 | 80 | 0 | 42.86 | 14.29 | fALS Up |
| expression | regulates | | | | | |
| (Transcription) | transcription of | | | | | |
| | genes involved | | | | | |
| | in differentiation | | | | | |
| | of myeloid cells | | | | | |
| Gene | RUNX3 | 80 | 0 | 57.14 | 42.86 | fALS Up |
| expression | regulates | | | | | |
| (Transcription) | CDKN1A | | | | | |
| | transcription | | | | | |
| Gene | TP53 Regulates | 100 | 0 | 42.86 | 57.14 | fALS Up |
| expression | Transcription of | | | | | |
| (Transcription) | Cell Cycle | | | | | |
| | Genes | | | | | |
| Gene | TP53 Regulates | 80 | 20 | 42.86 | 57.14 | fALS Up |
| expression | Transcription of | | | | | |
| (Transcription) | Genes Involved | | | | | |
| | in G2 Cell Cycle | | | | | |
| | Arrest | | | | | |
| Gene | Transcriptional | 80 | 20 | 42.86 | 57.14 | fALS Up |
| expression | Regulation by | | | | | |
| (Transcription) | MECP2 | | | | | |
| Gene | Transcriptional | 100 | 0 | 57.14 | 42.86 | fALS Up |
| expression | regulation by the | | | | | |
| (Transcription) | AP-2 (TFAP2) | | | | | |
| | family of | | | | | |
| | transcription | | | | | |
| | factors | | | | | |
| Gene | Transcriptional | 80 | 20 | 28.57 | 71.43 | fALS Up |
| expression | Regulation by | | | | | |
| (Transcription) | VENTX | | | | | |
| Gene | FOXO-mediated | 100 | 0 | 85.71 | 14.29 | fALS Up; |
| expression | transcription of | | | | | sALS Up |
| (Transcription) | cell cycle genes | | | | | |
| Gene | RNA | 20 | 80 | 14.29 | 85.71 | sALS |
| expression | Polymerase I | | | | | Down |
| (Transcription) | Transcription | | | | | |
| | Initiation | | | | | |
| Gene | RNA | 20 | 80 | 14.29 | 85.71 | sALS |
| expression | Polymerase I | | | | | Down |
| (Transcription) | Transcription | | | | | |
| | Termination | | | | | |
| Gene | RNA | 80 | 20 | 14.29 | 85.71 | sALS |
| expression | Polymerase II | | | | | Down |
| (Transcription) | Transcription | | | | | |
| | Termination | | | | | |
| Gene | RNA | 20 | 80 | 14.29 | 85.71 | sALS |
| expression | Polymerase III | | | | | Down |
| (Transcription) | Abortive And | | | | | |
| | Retractive | | | | | |
| | Initiation | | | | | |
| Gene | RNA | 0 | 80 | 0 | 100 | sALS |
| expression | Polymerase III | | | | | Down |
| (Transcription) | Chain | | | | | |
| | Elongation | | | | | |
| Gene | RNA | 20 | 80 | 14.29 | 85.71 | sALS |
| expression | Polymerase III | | | | | Down |
| (Transcription) | Transcription | | | | | |
| Gene | RNA | 20 | 80 | 14.29 | 85.71 | sALS |
| expression | Polymerase III | | | | | Down |
| (Transcription) | Transcription | | | | | |
| | Initiation | | | | | |
| Gene | RNA | 0 | 80 | 14.29 | 85.71 | sALS |
| expression | Polymerase III | | | | | Down |
| (Transcription) | Transcription | | | | | |
| | Initiation From | | | | | |
| | Type 1 Promoter | | | | | |
| Gene | RNA | 0 | 80 | 14.29 | 85.71 | sALS |
| expression | Polymerase III | | | | | Down |
| (Transcription) | Transcription | | | | | |
| | Initiation From | | | | | |
| | Type 2 Promoter | | | | | |
| Gene | RNA | 40 | 60 | 0 | 100 | sALS |
| expression | Polymerase III | | | | | Down |
| (Transcription) | Transcription | | | | | |
| | Initiation From | | | | | |
| | Type 3 Promoter | | | | | |
| Gene | RNA | 0 | 100 | 14.29 | 85.71 | sALS |
| expression | Polymerase III | | | | | Down |
| (Transcription) | Transcription | | | | | |
| | Termination | | | | | |
| Gene | FOXO-mediated | 60 | 40 | 85.71 | 14.29 | sALS Up |
| expression | transcription of | | | | | |
| (Transcription) | oxidative stress, | | | | | |
| | metabolic and | | | | | |
| | neuronal genes | | | | | |
| Gene | Regulation of | 60 | 40 | 85.71 | 14.29 | sALS Up |
| expression | TP53 Activity | | | | | |
| (Transcription) | through | | | | | |
| | Methylation | | | | | |
| Hemostasis | Common | 80 | 20 | 42.86 | 57.14 | fALS Up |
| | Pathway of | | | | | |
| | Fibrin Clot | | | | | |
| | Formation | | | | | |
| Hemostasis | Extrinsic | 80 | 20 | 14.29 | 57.14 | fALS Up |
| | Pathway of | | | | | |
| | Fibrin Clot | | | | | |
| | Formation | | | | | |
| Hemostasis | Formation of | 80 | 20 | 42.86 | 57.14 | fALS Up |
| | Fibrin Clot | | | | | |
| | (Clotting | | | | | |
| | Cascade) | | | | | |
| Hemostasis | Hemostasis | 100 | 0 | 57.14 | 42.86 | fALS Up |
| Hemostasis | Intrinsic | 80 | 20 | 42.86 | 57.14 | fALS Up |
| | Pathway of | | | | | |
| | Fibrin Clot | | | | | |
| | Formation | | | | | |
| Hemostasis | Platelet | 80 | 20 | 57.14 | 42.86 | fALS Up |
| | activation, | | | | | |
| | signaling and | | | | | |
| | aggregation | | | | | |
| Hemostasis | Platelet | 80 | 0 | 28.57 | 57.14 | fALS Up |
| | Adhesion to | | | | | |
| | exposed | | | | | |
| | collagen | | | | | |
| Hemostasis | Platelet | 80 | 20 | 71.43 | 28.57 | fALS Up |
| | Aggregation | | | | | |
| | (Plug | | | | | |
| | Formation) | | | | | |
| Hemostasis | Platelet | 100 | 0 | 71.43 | 28.57 | fALS Up |
| | degranulation | | | | | |
| Hemostasis | Response to | 100 | 0 | 71.43 | 28.57 | fALS Up |
| | elevated platelet | | | | | |
| | cytosolic Ca²⁺ | | | | | |
| Hemostasis | Tie2 Signaling | 100 | 0 | 71.43 | 28.57 | fALS Up |
| Hemostasis, | GRB2:SOS | 80 | 0 | 71.43 | 28.57 | fALS Up |
| Signal | provides linkage | | | | | |
| Transduction | to MAPK | | | | | |
| | signaling for | | | | | |
| | Integrins | | | | | |
| Immune | Antigen | 100 | 0 | 42.86 | 57.14 | fALS Up |
| System | activates B Cell | | | | | |
| | Receptor (BCR) | | | | | |
| | leading to | | | | | |
| | generation of | | | | | |
| | second | | | | | |
| | messengers | | | | | |
| Immune | Antigen | 80 | 20 | 57.14 | 42.86 | fALS Up |
| System | processing- | | | | | |
| | Cross | | | | | |
| | presentation | | | | | |
| Immune | ER-Phagosome | 80 | 20 | 57.14 | 42.86 | fALS Up |
| System | pathway | | | | | |
| Immune | Antimicrobial | 80 | 20 | 42.86 | 57.14 | fALS Up |
| System | peptides | | | | | |
| Immune | CD28 co- | 80 | 20 | 42.86 | 57.14 | fALS Up |
| System | stimulation | | | | | |
| Immune | Interleukin-1 | 80 | 20 | 57.14 | 42.86 | fALS Up |
| System | family signaling | | | | | |
| Immune | DAP12 | 100 | 0 | 42.86 | 57.14 | fALS Up |
| System | interactions | | | | | |
| Immune | DAP12 | 100 | 0 | 42.86 | 57.14 | fALS Up |
| System | signaling | | | | | |
| Immune | Interleukin-1 | 80 | 20 | 57.14 | 42.86 | fALS Up |
| System | signaling | | | | | |
| Immune | Interleukin-17 | 80 | 20 | 57.14 | 42.86 | fALS Up |
| System | signaling | | | | | |
| Immune | Interleukin-18 | 80 | 20 | 42.86 | 42.86 | fALS Up |
| System | signaling | | | | | |
| Immune | NIK- | 60 | 40 | 14.29 | 85.71 | sALS |
| System | →noncanonical | | | | | Down |
| | NF-kB signaling | | | | | |
| Immune | Interleukin-33 | 60 | 0 | 85.71 | 14.29 | sALS Up |
| System | signaling | | | | | |
| Immune | DDX58-IFIH1- | 80 | 20 | 42.86 | 57.14 | fALS Up |
| System | mediated | | | | | |
| | induction of | | | | | |
| | interferon-alpha- | | | | | |
| | beta | | | | | |
| Immune | TRAF6 | 80 | 0 | 57.14 | 42.86 | fALS Up |
| System | mediated NF-kB | | | | | |
| | activation | | | | | |
| Immune | Interferon alpha- | 80 | 20 | 42.86 | 57.14 | fALS Up |
| System | beta signaling | | | | | |
| Immune | Interferon | 80 | 20 | 42.86 | 57.14 | fALS Up |
| System | Signaling | | | | | |
| Immune | Interleukin | 100 | 0 | 42.86 | 57.14 | fALS Up |
| System | receptor SHC | | | | | |
| | signaling | | | | | |
| Immune | ZBP1(DAI) | 80 | 20 | 57.14 | 42.86 | fALS Up |
| System | mediated | | | | | |
| | induction of type | | | | | |
| | I IFNs | | | | | |
| Immune | Dectin-1 | 60 | 40 | 14.29 | 85.71 | sALS |
| System | mediated | | | | | Down |
| | noncanonical | | | | | |
| | NF-KB signaling | | | | | |
| Immune | Interleukin-10 | 80 | 20 | 57.14 | 42.86 | fALS Up |
| System | signaling | | | | | |
| Immune | NOD1-2 | 60 | 40 | 14.29 | 85.71 | sALS |
| System | Signaling | | | | | Down |
| | Pathway | | | | | |
| Immune | Nucleotide- | 40 | 60 | 14.29 | 85.71 | sALS |
| System | binding domain, | | | | | Down |
| | leucine rich | | | | | |
| | repeat | | | | | |
| | containing | | | | | |
| | receptor (NLR) | | | | | |
| | signaling | | | | | |
| | pathways | | | | | |
| Immune | Interleukin-3, | 100 | 0 | 42.86 | 57.14 | fALS Up |
| System | Interleukin-5 | | | | | |
| | and GM-CSF | | | | | |
| | signaling | | | | | |
| Immune | LRR FLII- | 80 | 0 | 57.14 | 28.57 | fALS Up |
| System | interacting | | | | | |
| | protein 1 | | | | | |
| | (LRRFIP1) | | | | | |
| | activates type I | | | | | |
| | IFN production | | | | | |
| Immune | Fc epsilon | 80 | 20 | 28.57 | 71.43 | fALS Up |
| System | receptor | | | | | |
| | (FCERI) | | | | | |
| | signaling | | | | | |
| Immune | Metal | 100 | 0 | 57.14 | 14.29 | fALS Up |
| System | sequestration by | | | | | |
| | antimicrobial | | | | | |
| | proteins | | | | | |
| Immune | FCERI mediated | 80 | 20 | 28.57 | 71.43 | fALS Up |
| System | Ca+2 | | | | | |
| | mobilization | | | | | |
| Immune | FCERI mediated | 80 | 20 | 28.57 | 71.43 | fALS Up |
| System | MAPK | | | | | |
| | activation | | | | | |
| Immune | Regulation of | 100 | 0 | 71.43 | 28.57 | fALS Up |
| System | IFNA signaling | | | | | |
| Immune | FCERI mediated | 80 | 20 | 71.43 | 28.57 | fALS Up |
| System | NF-kB | | | | | |
| | activation | | | | | |
| Immune | Role of LAT2- | 80 | 20 | 57.14 | 42.86 | fALS Up |
| System | NTAL-LAB on | | | | | |
| | calcium | | | | | |
| | mobilization | | | | | |
| Immune | TNFs bind their | 80 | 20 | 57.14 | 28.57 | fALS Up |
| System | physiological | | | | | |
| | receptors | | | | | |
| Immune | Activation of C3 | 80 | 0 | 28.57 | 28.57 | fALS Up |
| System | and C5 | | | | | |
| Immune | Complement | 100 | 0 | 28.57 | 71.43 | fALS Up |
| System | cascade | | | | | |
| Immune | Regulation of | 100 | 0 | 42.86 | 57.14 | fALS Up |
| System | Complement | | | | | |
| | cascade | | | | | |
| Immune | Classical | 80 | 0 | 14.29 | 85.71 | fALS Up; |
| System | antibody- | | | | | sALS |
| | mediated | | | | | Down |
| | complement | | | | | |
| | activation | | | | | |
| Immune | IKK complex | 100 | 0 | 42.86 | 42.86 | fALS Up |
| System | recruitment | | | | | |
| | mediated by | | | | | |
| | RIP1 | | | | | |
| Immune | MAP kinase | 80 | 20 | 85.71 | 14.29 | fALS Up |
| System | activation | | | | | |
| Immune | MyD88:MAL(T | 80 | 20 | 71.43 | 28.57 | fALS Up |
| System | IRAP) cascade | | | | | |
| | initiated on | | | | | |
| | plasma | | | | | |
| | membrane | | | | | |
| Immune | Regulation of | 80 | 0 | 71.43 | 28.57 | fALS Up |
| System | TLR by | | | | | |
| | endogenous | | | | | |
| | ligand | | | | | |
| Immune | Toll Like | 80 | 20 | 71.43 | 28.57 | fALS Up |
| System | Receptor 2 | | | | | |
| | (TLR2) Cascade | | | | | |
| Immune | Toll Like | 80 | 20 | 28.57 | 71.43 | fALS Up |
| System | Receptor 4 | | | | | |
| | (TLR4) Cascade | | | | | |
| Immune | Toll Like | 80 | 20 | 71.43 | 28.57 | fALS Up |
| System | Receptor | | | | | |
| | TLR1:TLR2 | | | | | |
| | Cascade | | | | | |
| Immune | Toll Like | 80 | 20 | 71.43 | 28.57 | fALS Up |
| System | Receptor | | | | | |
| | TLR6:TLR2 | | | | | |
| | Cascade | | | | | |
| Immune | TRIF-mediated | 100 | 0 | 28.57 | 57.14 | fALS Up |
| System | programmed cell | | | | | |
| | death | | | | | |
| Metabolism | Glyoxylate | 0 | 80 | 42.86 | 57.14 | fALS |
| | metabolism and | | | | | Down |
| | glycine | | | | | |
| | degradation | | | | | |
| Metabolism | A | 80 | 20 | 57.14 | 42.86 | fALS Up |
| | tetrasaccharide | | | | | |
| | linker sequence | | | | | |
| | is required for | | | | | |
| | GAG synthesis | | | | | |
| Metabolism | Androgen | 80 | 0 | 28.57 | 42.86 | fALS Up |
| | biosynthesis | | | | | |
| Metabolism | Chondroitin | 80 | 20 | 57.14 | 42.86 | fALS Up |
| | sulfate-dermatan | | | | | |
| | sulfate | | | | | |
| | metabolism | | | | | |
| Metabolism | Glycosaminoglycan | 80 | 20 | 57.14 | 42.86 | fALS Up |
| | metabolism | | | | | |
| Metabolism | Heparan sulfate- | 80 | 20 | 42.86 | 57.14 | fALS Up |
| | heparin (HS- | | | | | |
| | GAG) | | | | | |
| | metabolism | | | | | |
| Metabolism | HS-GAG | 80 | 20 | 42.86 | 57.14 | fALS Up |
| | biosynthesis | | | | | |
| Metabolism | HS-GAG | 80 | 20 | 42.86 | 57.14 | fALS Up |
| | degradation | | | | | |
| Metabolism | Metabolism of | 80 | 20 | 57.14 | 42.86 | fALS Up |
| | carbohydrates | | | | | |
| Metabolism | Metabolism of | 80 | 20 | 57.14 | 42.86 | fALS Up |
| | fat-soluble | | | | | |
| | vitamins | | | | | |
| Metabolism | Xenobiotics | 80 | 0 | 57.14 | 42.86 | fALS Up |
| Metabolism | Arachidonic acid | 60 | 40 | 14.29 | 85.71 | sALS |
| | metabolism | | | | | Down |
| Metabolism | Complex I | 0 | 80 | 14.29 | 85.71 | sALS |
| | biogenesis | | | | | Down |
| Metabolism | Cytosolic | 20 | 40 | 0 | 85.71 | sALS |
| | sulfonation of | | | | | Down |
| | small molecules | | | | | |
| Metabolism | Glycerophospho | 60 | 40 | 14.29 | 85.71 | sALS |
| | lipid | | | | | Down |
| | biosynthesis | | | | | |
| Metabolism | Phase II- | 20 | 60 | 14.29 | 85.71 | sALS |
| | Conjugation of | | | | | Down |
| | compounds | | | | | |
| Metabolism | Phospholipid | 40 | 60 | 14.29 | 85.71 | sALS |
| | metabolism | | | | | Down |
| Metabolism | Respiratory | 20 | 80 | 14.29 | 85.71 | sALS |
| | electron | | | | | Down |
| | transport | | | | | |
| Metabolism | Respiratory | 20 | 80 | 14.29 | 85.71 | sALS |
| | electron | | | | | Down |
| | transport, ATP | | | | | |
| | synthesis by | | | | | |
| | chemiosmotic | | | | | |
| | coupling, and | | | | | |
| | heat production | | | | | |
| | by uncoupling | | | | | |
| | proteins | | | | | |
| Metabolism | Synthesis of | 40 | 20 | 14.29 | 85.71 | sALS |
| | Prostaglandins | | | | | Down |
| | (PG) and | | | | | |
| | Thromboxanes | | | | | |
| | (TX) | | | | | |
| Metabolism of | CREB3 factors | 0 | 100 | 28.57 | 71.43 | fALS |
| proteins | activate genes | | | | | Down |
| Metabolism of | Attachment of | 80 | 0 | 28.57 | 28.57 | fALS Up |
| proteins | GPI anchor to | | | | | |
| | uPAR | | | | | |
| Metabolism of | IRE1alpha | 80 | 20 | 57.14 | 42.86 | fALS Up |
| proteins | activates | | | | | |
| | chaperones | | | | | |
| Metabolism of | Metalloprotease | 80 | 20 | 28.57 | 71.43 | fALS Up |
| proteins | DUBs | | | | | |
| Metabolism of | Post- | 80 | 20 | 57.14 | 42.86 | fALS Up |
| proteins | translational | | | | | |
| | protein | | | | | |
| | phosphorylation | | | | | |
| Metabolism of | Unfolded | 80 | 20 | 57.14 | 42.86 | fALS Up |
| proteins | Protein | | | | | |
| | Response (UPR) | | | | | |
| Metabolism of | XBP1(S) | 80 | 20 | 57.14 | 42.86 | fALS Up |
| proteins | activates | | | | | |
| | chaperone genes | | | | | |
| Metabolism of | Cooperation of | 40 | 40 | 14.29 | 85.71 | sALS |
| proteins | Prefoldin and | | | | | Down |
| | TriC-CCT in | | | | | |
| | actin and tubulin | | | | | |
| | folding | | | | | |
| Metabolism of | Gamma | 60 | 40 | 14.29 | 85.71 | sALS |
| proteins | carboxylation, | | | | | Down |
| | hypusine | | | | | |
| | formation and | | | | | |
| | arylsulfatase | | | | | |
| | activation | | | | | |
| Metabolism of | Post-chaperonin | 60 | 40 | 14.29 | 85.71 | sALS |
| proteins | tubulin folding | | | | | Down |
| | pathway | | | | | |
| Metabolism of | Metabolism of | 20 | 80 | 14.29 | 85.71 | sALS |
| RNA | non-coding | | | | | Down |
| | RNA | | | | | |
| Metabolism of | mRNA Splicing- | 60 | 40 | 14.29 | 85.71 | sALS |
| RNA | Minor Pathway | | | | | Down |
| Metabolism of | snRNP | 20 | 80 | 14.29 | 85.71 | sALS |
| RNA | Assembly | | | | | Down |
| Metabolism of | Transport of | 40 | 60 | 14.29 | 85.71 | sALS |
| RNA | Mature mRNA | | | | | Down |
| | derived from an | | | | | |
| | Intron- | | | | | |
| | Containing | | | | | |
| | Transcript | | | | | |
| Metabolism of | Transport of | 40 | 60 | 14.29 | 85.71 | sALS |
| RNA | Mature | | | | | Down |
| | Transcript to | | | | | |
| | Cytoplasm | | | | | |
| Metabolism of | HuR (ELAVL1) | 40 | 20 | 85.71 | 0 | sALS Up |
| RNA | binds and | | | | | |
| | stabilizes mRNA | | | | | |
| Metabolism of | Regulation of | 20 | 80 | 85.71 | 0 | sALS Up |
| RNA | mRNA stability | | | | | |
| | by proteins that | | | | | |
| | bind AU-rich | | | | | |
| | elements | | | | | |
| Metabolism, | Retinoid | 80 | 20 | 57.14 | 42.86 | fALS Up |
| Signal | metabolism and | | | | | |
| Transduction | transport | | | | | |
| Muscle | Phase 0-rapid | 40 | 60 | 14.29 | 85.71 | sALS |
| contraction | depolarisation | | | | | Down |
| Organelle | Cilium | 20 | 80 | 28.57 | 71.43 | fALS |
| biogenesis and | Assembly | | | | | Down |
| maintenance | | | | | | |
| Organelle | Organelle | 20 | 80 | 28.57 | 71.43 | fALS |
| biogenesis and | biogenesis and | | | | | Down |
| maintenance | maintenance | | | | | |
| Organelle | Intraflagellar | 20 | 80 | 14.29 | 85.71 | fALS |
| biogenesis and | transport | | | | | Down; |
| maintenance | | | | | | ALS |
| | | | | | | Down |
| Programmed | Activation of | 80 | 0 | 42.86 | 28.57 | fALS Up |
| Cell Death | BAD and | | | | | |
| | translocation to | | | | | |
| | mitochondria | | | | | |
| Programmed | Activation of | 80 | 20 | 42.86 | 57.14 | fALS Up |
| Cell Death | BH3-only | | | | | |
| | proteins | | | | | |
| Programmed | Apoptosis | 80 | 20 | 42.86 | 42.86 | fALS Up |
| Cell Death | induced DNA | | | | | |
| | fragmentation | | | | | |
| Programmed | CASP8 activity | 80 | 20 | 14.29 | 71.43 | fALS Up |
| Cell Death | is inhibited | | | | | |
| Programmed | Regulated | 100 | 0 | 57.14 | 42.86 | fALS Up |
| Cell Death | Necrosis | | | | | |
| Programmed | Regulation of | 80 | 20 | 14.29 | 71.43 | fALS Up |
| Cell Death | necroptotic cell | | | | | |
| | death | | | | | |
| Programmed | RIPK1-mediated | 100 | 0 | 57.14 | 42.86 | fALS Up |
| Cell Death | regulated | | | | | |
| | necrosis | | | | | |
| Programmed | Apoptotic | 100 | 0 | 85.71 | 14.29 | fALS Up; |
| Cell Death | execution phase | | | | | sALS Up |
| Programmed | Release of | 20 | 40 | 0 | 85.71 | sALS |
| Cell Death | apoptotic factors | | | | | Down |
| | from the | | | | | |
| | mitochondria | | | | | |
| Protein | Mitochondrial | 40 | 60 | 14.29 | 85.71 | sALS |
| localization | protein import | | | | | Down |
| Signal | Insulin receptor | 20 | 80 | 28.57 | 71.43 | fALS |
| Transduction | recycling | | | | | Down |
| Signal | Signaling by | 20 | 80 | 42.86 | 57.14 | fALS |
| Transduction | NTRK2 (TRKB) | | | | | Down |
| Signal | AKT | 80 | 20 | 57.14 | 42.86 | fALS Up |
| Transduction | phosphorylates | | | | | |
| | targets in the | | | | | |
| | cytosol | | | | | |
| Signal | Death Receptor | 80 | 20 | 57.14 | 42.86 | fALS Up |
| Transduction | Signalling | | | | | |
| Signal | Downregulation | 80 | 20 | 57.14 | 28.57 | fALS Up |
| Transduction | of ERBB4 | | | | | |
| | signaling | | | | | |
| Signal | G alpha (i) | 80 | 20 | 42.86 | 57.14 | fALS Up |
| Transduction | signalling events | | | | | |
| Signal | MET activates | 80 | 20 | 100 | 0 | fALS Up |
| Transduction | RAP1 and | | | | | |
| | RAC1 | | | | | |
| Signal | MET activates | 80 | 20 | 71.43 | 28.57 | fALS Up |
| Transduction | RAS signaling | | | | | |
| Signal | MET interacts | 80 | 20 | 57.14 | 14.29 | fALS Up |
| Transduction | with TNS | | | | | |
| | proteins | | | | | |
| Signal | MET promotes | 80 | 20 | 71.43 | 28.57 | fALS Up |
| Transduction | cell motility | | | | | |
| Signal | Negative | 80 | 20 | 57.14 | 42.86 | fALS Up |
| Transduction | regulation of | | | | | |
| | MET activity | | | | | |
| Signal | Nuclear | 100 | 0 | 57.14 | 42.86 | fALS Up |
| Transduction | signaling by | | | | | |
| | ERBB4 | | | | | |
| Signal | Regulation of | 80 | 20 | 28.57 | 71.43 | fALS Up |
| Transduction | FZD by | | | | | |
| | ubiquitination | | | | | |
| Signal | RHO GTPases | 80 | 20 | 57.14 | 42.86 | fALS Up |
| Transduction | Activate | | | | | |
| | NADPH | | | | | |
| | Oxidases | | | | | |
| Signal | Serotonin | 80 | 20 | 42.86 | 57.14 | fALS Up |
| Transduction | receptors | | | | | |
| Signal | Signaling by | 80 | 20 | 42.86 | 57.14 | fALS Up |
| Transduction | ERBB2 | | | | | |
| Signal | Signaling by | 100 | 0 | 71.43 | 28.57 | fALS Up |
| Transduction | ERBB4 | | | | | |
| Signal | Signaling by | 80 | 20 | 71.43 | 28.57 | fALS Up |
| Transduction | MET | | | | | |
| Signal | TNFR1-induced | 80 | 20 | 57.14 | 42.86 | fALS Up |
| Transduction | NFkappaB | | | | | |
| | signaling | | | | | |
| | pathway | | | | | |
| Signal | TNFR1-induced | 80 | 20 | 42.86 | 28.57 | fALS Up |
| Transduction | proapoptotic | | | | | |
| | signaling | | | | | |
| Signal | Visual | 80 | 20 | 57.14 | 42.86 | fALS Up |
| Transduction | phototransduction | | | | | |
| Signal | Erythropoietin | 80 | 0 | 85.71 | 14.29 | fALS Up; |
| Transduction | activates | | | | | sALS Up |
| | Phosphoinositide- | | | | | |
| | 3-kinase | | | | | |
| | (PI3K) | | | | | |
| Signal | Signaling by | 80 | 20 | 85.71 | 14.29 | fALS Up; |
| Transduction | Erythropoietin | | | | | sALS Up |
| Signal | Degradation of | 60 | 0 | 14.29 | 85.71 | sALS |
| Transduction | DVL | | | | | Down |
| Signal | Downstream | 80 | 20 | 14.29 | 85.71 | sALS |
| Transduction | signaling of | | | | | Down |
| | activated FGFR3 | | | | | |
| Signal | Downstream | 80 | 20 | 14.29 | 85.71 | sALS |
| Transduction | signaling of | | | | | Down |
| | activated FGFR4 | | | | | |
| Signal | FGFR1 ligand | 40 | 60 | 14.29 | 85.71 | sALS |
| Transduction | binding and | | | | | Down |
| | activation | | | | | |
| Signal | FGFR2 ligand | 20 | 80 | 14.29 | 85.71 | sALS |
| Transduction | binding and | | | | | Down |
| | activation | | | | | |
| Signal | FGFR2b ligand | 20 | 80 | 14.29 | 85.71 | sALS |
| Transduction | binding and | | | | | Down |
| | activation | | | | | |
| Signal | FGFR2c ligand | 40 | 60 | 14.29 | 85.71 | sALS |
| Transduction | binding and | | | | | Down |
| | activation | | | | | |
| Signal | FGFR3 ligand | 60 | 40 | 14.29 | 85.71 | sALS |
| Transduction | binding and | | | | | Down |
| | activation | | | | | |
| Signal | FGFR3b ligand | 60 | 40 | 0 | 85.71 | sALS |
| Transduction | binding and | | | | | Down |
| | activation | | | | | |
| Signal | FGFR3c ligand | 60 | 40 | 14.29 | 85.71 | sALS |
| Transduction | binding and | | | | | Down |
| | activation | | | | | |
| Signal | FGFR4 ligand | 40 | 60 | 14.29 | 85.71 | sALS |
| Transduction | binding and | | | | | Down |
| | activation | | | | | |
| Signal | FGFRL1 | 60 | 40 | 0 | 100 | sALS |
| Transduction | modulation of | | | | | Down |
| | FGFR1 | | | | | |
| | signaling | | | | | |
| Signal | FRS-mediated | 60 | 40 | 14.29 | 85.71 | sALS |
| Transduction | FGFR3 | | | | | Down |
| | signaling | | | | | |
| Signal | FRS-mediated | 40 | 60 | 14.29 | 85.71 | sALS |
| Transduction | FGFR4 | | | | | Down |
| | signaling | | | | | |
| Signal | Frs2-mediated | 60 | 40 | 14.29 | 85.71 | sALS |
| Transduction | activation | | | | | Down |
| Signal | Negative | 40 | 60 | 14.29 | 85.71 | sALS |
| Transduction | regulation of | | | | | Down |
| | FGFR4 | | | | | |
| | signaling | | | | | |
| Signal | Negative | 20 | 40 | 0 | 100 | sALS |
| Transduction | regulation of | | | | | Down |
| | TCF-dependent | | | | | |
| | signaling by | | | | | |
| | DVL-interacting | | | | | |
| | proteins | | | | | |
| Signal | PCP-CE | 60 | 40 | 14.29 | 85.71 | sALS |
| Transduction | pathway | | | | | Down |
| Signal | Phospholipase | 40 | 60 | 14.29 | 85.71 | sALS |
| Transduction | C-mediated | | | | | Down |
| | cascade; FGFR2 | | | | | |
| Signal | Phospholipase | 60 | 40 | 14.29 | 85.71 | sALS |
| Transduction | C-mediated | | | | | Down |
| | cascade; FGFR3 | | | | | |
| Signal | Phospholipase | 40 | 60 | 14.29 | 85.71 | sALS |
| Transduction | C-mediated | | | | | Down |
| | cascade; FGFR4 | | | | | |
| Signal | PI-3K | 40 | 60 | 14.29 | 85.71 | sALS |
| Transduction | cascade:FGFR4 | | | | | Down |
| Signal | Prolonged ERK | 60 | 40 | 14.29 | 85.71 | sALS |
| Transduction | activation events | | | | | Down |
| Signal | Retrograde | 60 | 20 | 0 | 100 | sALS |
| Transduction | neurotrophin | | | | | Down |
| | signalling | | | | | |
| Signal | SHC-mediated | 80 | 20 | 14.29 | 85.71 | sALS |
| Transduction | cascade:FGFR1 | | | | | Down |
| Signal | SHC-mediated | 80 | 20 | 14.29 | 85.71 | sALS |
| Transduction | cascade:FGFR2 | | | | | Down |
| Signal | SHC-mediated | 80 | 20 | 14.29 | 85.71 | sALS |
| Transduction | cascade:FGFR3 | | | | | Down |
| Signal | SHC-mediated | 80 | 20 | 14.29 | 85.71 | sALS |
| Transduction | cascade:FGFR4 | | | | | Down |
| Signal | Signaling by | 60 | 40 | 14.29 | 85.71 | sALS |
| Transduction | FGFR | | | | | Down |
| Signal | Signaling by | 60 | 40 | 14.29 | 85.71 | sALS |
| Transduction | FGFR3 | | | | | Down |
| Signal | Signaling by | 60 | 40 | 14.29 | 85.71 | sALS |
| Transduction | FGFR4 | | | | | Down |
| Signal | TCF dependent | 60 | 40 | 14.29 | 85.71 | sALS |
| Transduction | signaling in | | | | | Down |
| | response to | | | | | |
| | WNT | | | | | |
| Signal | WNT mediated | 20 | 20 | 0 | 85.71 | sALS |
| Transduction | activation of | | | | | Down |
| | DVL | | | | | |
| Signal | MET activates | 60 | 20 | 100 | 0 | sALS Up |
| Transduction | PTPN11 | | | | | |
| Signal | RHO GTPases | 20 | 20 | 85.71 | 14.29 | sALS Up |
| Transduction | activate IQGAPs | | | | | |
| Signal | RHO GTPases | 80 | 20 | 85.71 | 14.29 | sALS Up |
| Transduction | Activate WASPs | | | | | |
| | and WAVEs | | | | | |
| Signal | Signaling by | 100 | 0 | 85.71 | 14.29 | sALS Up |
| Transduction | Hippo | | | | | |
| Signal | Signaling by | 60 | 40 | 85.71 | 14.29 | sALS Up |
| Transduction | VEGF | | | | | |
| Transport of | Transferrin | 20 | 80 | 28.57 | 71.43 | fALS |
| small | endocytosis and | | | | | Down |
| molecules | recycling | | | | | |
| Transport of | ABC-family | 80 | 20 | 71.43 | 28.57 | fALS Up |
| small | proteins | | | | | |
| molecules | mediated | | | | | |
| | transport | | | | | |
| Transport of | Ion channel | 60 | 40 | 14.29 | 85.71 | sALS |
| small | transport | | | | | Down |
| molecules | | | | | | |
| Transport of | VLDL clearance | 40 | 20 | 85.71 | 14.29 | sALS Up |
| small | | | | | | |
| molecules | | | | | | |
| Vesicle- | Binding and | 80 | 20 | 57.14 | 42.86 | fALS Up |
| mediated | Uptake of | | | | | |
| transport | Ligands by | | | | | |
| | Scavenger | | | | | |
| | Receptors | | | | | |
| Vesicle- | Scavenging by | 80 | 20 | 57.14 | 42.86 | fALS Up |
| mediated | Class A | | | | | |
| transport | Receptors | | | | | |

Dysregulated pathways in diMN transcriptomics comparisons

| Main | | | | |
|---|---|---|---|---|
| biological | | fALS | sALS | |
| process | Pathway | (iPanda) | (iPanda) | Direction |
| Developmental | Regulation of signaling by NODAL | −0.0022 | −0.0111 | sALS |
| Biology | | | | down |
| Digestion and | Digestion | 0.0132 | 0.0001 | fALS up |
| absorption | | | | |
| Digestion and | Digestion and absorption | 0.0121 | 0.0001 | fALS up |
| absorption | | | | |
| Digestion and | Digestion of dietary lipid | 0.0209 | 0 | fALS up |
| absorption | | | | |
| Disease | Diseases associated with visual transduction | 0 | 0.0255 | sALS up |
| Disease | Diseases of the neuronal system | 0 | 0.0255 | sALS up |
| Disease | Retinoid cycle disease events | 0 | 0.0255 | sALS up |
| Disease, Signal | Biosynthesis of A2E, implicated in retinal | 0 | 0.0255 | sALS up |
| Transduction | degradation | | | |
| Immune | Activation of C3 and C5 | 0.0134 | 0.0029 | fALS up |
| System | | | | |
| Immune | Alpha-defensins | 0.0002 | −0.0104 | sALS |
| System | | | | down |
| Immune | Ficolins bind to repetitive carbohydrate | −0.0187 | 0 | fALS |
| System | structures on the target cell surface | | | down |
| Immune | Lectin pathway of complement activation | −0.0125 | 0 | fALS |
| System | | | | down |
| Immune | OAS antiviral response | 0.0351 | 0.0189 | fALS up, |
| System | | | | sALS up |
| Immune | CREB phosphorylation | 0.0186 | 0.0219 | fALS up, |
| System, Signal | | | | sALS up |
| Transduction | | | | |
| Metabolism | Acyl chain remodeling of DAG and TAG | 0.0272 | 0 | fALS up |
| Metabolism | Blood group systems biosynthesis | 0.0305 | 0 | fALS up |
| Metabolism | Lipid particle organization | 0.0004 | −0.0101 | sALS |
| | | | | down |
| Metabolism | PAOs oxidise polyamines to amines | 0.0109 | 0.0007 | fALS up |
| Metabolism | Rhesus blood group biosynthesis | 0.0305 | 0 | fALS up |
| Metabolism of | Activation of the mRNA upon binding of the | 0.0005 | 0.0137 | sALS up |
| proteins | cap-binding complex and e1Fs, and | | | |
| | subsequent binding to 43S | | | |
| Metabolism of | Cap-dependent Translation Initiation | 0.0004 | 0.0114 | sALS up |
| proteins | | | | |
| Metabolism of | Eukaryotic Translation Initiation | 0.0004 | 0.0111 | sALS up |
| proteins | | | | |
| Metabolism of | Formation of the ternary complex, and | 0.0005 | 0.0161 | sALS up |
| proteins | subsequently, the 43S complex | | | |
| Metabolism of | GTP hydrolysis and joining of the 60S | 0.0003 | 0.0104 | sALS up |
| proteins | ribosomal subunit | | | |
| Metabolism of | Ribosomal scanning and start codon | 0.0005 | 0.0141 | sALS up |
| proteins | recognition | | | |
| Metabolism of | Translation initiation complex formation | 0.0005 | 0.0141 | sALS up |
| proteins | | | | |
| Metabolism of | Major pathway of rRNA processing in the | 0.0005 | 0.0122 | sALS up |
| RNA | nucleolus and cytosol | | | |
| Metabolism of | rRNA processing | 0.0005 | 0.0122 | sALS up |
| RNA | | | | |
| Metabolism of | rRNA processing in the nucleus and cytosol | 0.0005 | 0.0122 | sALS up |
| RNA | | | | |
| Neuronal | Neurexins and neuroligins | 0.0003 | 0.0351 | sALS up |
| System | | | | |
| Neuronal | Protein-protein interactions at synapses | 0.0003 | 0.0281 | sALS up |
| System | | | | |
| Signal | Activation of the phototransduction cascade | 0 | −0.0135 | sALS |
| Transduction | | | | down |
| Signal | Serotonin receptors | −0.0134 | −0.003 | fALS |
| Transduction | | | | down |
| Transport of | Erythrocytes take up carbon dioxide and | 0 | −0.053 | sALS |
| small | release oxygen | | | down |
| molecules | | | | |
| Transport of | Erythrocytes take up oxygen and release | 0 | −0.053 | sALS |
| small | carbon dioxide | | | down |
| molecules | | | | |
| Transport of | O2—CO2 exchange in erythrocytes | 0 | −0.053 | sALS |
| small | | | | down |
| molecules | | | | |

Dysregulated pathways in diMN proteomics comparisons

| Main | | | | |
|---|---|---|---|---|
| biological | | fALS | sALS | |
| process | Pathway | (iPanda) | (iPanda) | Direction |
| Cell Cycle, | Meiosis | 0.0106 | −0.0006 | fALS up |
| Reproduction | | | | |
| Cell Cycle, | Meiotic recombination | 0.0154 | −0.0007 | fALS up |
| Reproduction | | | | |
| Cell-Cell | Adherens junctions interactions | −0.0106 | −0.0006 | fALS |
| communication | | | | down |
| Cell-Cell | Nectin-Necl trans heterodimerization | −0.0104 | −0.0003 | fALS |
| communication | | | | down |
| Developmental | Inactivation of CDC42 and RAC1 | −0.0026 | −0.0133 | sALS |
| Biology | | | | down |
| Developmental | Myogenesis | −0.0174 | 0 | fALS |
| Biology | | | | down |
| Developmental | NCAM signaling for neurite out-growth | −0.0104 | −0.0003 | fALS |
| Biology | | | | down |
| Developmental | NCAM1 interactions | −0.026 | −0.0005 | fALS |
| Biology | | | | down |
| Developmental | Neurofascin interactions | −0.037 | −0.0008 | fALS |
| Biology | | | | down |
| Developmental | NrCAM interactions | −0.0123 | −0.003 | fALS |
| Biology | | | | down |
| Developmental | Other semaphorin interactions | −0.0105 | 0 | fALS |
| Biology | | | | down |
| Disease | ABC transporter disorders | 0.0008 | 0.012 | sALS up |
| Disease | APOBEC3G mediated resistance to HIV-1 | 0.001 | 0.0326 | sALS up |
| | infection | | | |
| Disease | Assembly Of The HIV Virion | 0.0009 | 0.0304 | sALS up |
| Disease | Binding and entry of HIV virion | 0.0026 | 0.0912 | sALS up |
| Disease | Defective CFTR causes cystic fibrosis | 0.0008 | 0.012 | sALS up |
| Disease | Disorders of transmembrane transporters | 0.0008 | 0.012 | sALS up |
| Disease | Early Phase of HIV Life Cycle | 0.0006 | 0.0204 | sALS up |
| Disease | Hh mutants abrogate ligand secretion | 0 | 0.0214 | sALS up |
| Disease | Hh mutants that don't undergo autocatalytic | 0 | 0.0214 | sALS up |
| | processing are degraded by ERAD | | | |
| Disease | InlA-mediated entry of Listeria | −0.0339 | −0.0028 | fALS |
| | monocytogenes into host cells | | | down |
| Disease | Integration of provirus | 0.0004 | 0.0161 | sALS up |
| Disease | Listeria monocytogenes entry into host cells | −0.0109 | −0.0009 | fALS |
| | | | | down |
| Disease | Minus-strand DNA synthesis | 0.0026 | 0.0912 | sALS up |
| Disease | Plus-strand DNA synthesis | 0.0026 | 0.0912 | sALS up |
| Disease | Reverse Transcription of HIV RNA | 0.0026 | 0.0912 | sALS up |
| Disease | Uncoating of the HIV Virion | 0.0026 | 0.0912 | sALS up |
| DNA Repair | HDR through Homologous Recombination | 0.0202 | 0 | fALS up |
| | (HRR) | | | |
| DNA Repair | Homologous DNA Pairing and Strand | 0.0156 | 0 | fALS up |
| | Exchange | | | |
| DNA Repair | Presynaptic phase of homologous DNA | 0.0147 | 0 | fALS up |
| | pairing and strand exchange | | | |
| DNA Repair | Resolution of D-Loop Structures | 0.0213 | 0 | fALS up |
| DNA Repair | Resolution of D-loop Structures through | 0.0213 | 0 | fALS up |
| | Holliday Junction Intermediates | | | |
| DNA Repair | Resolution of D-loop Structures through | 0.0213 | 0 | fALS up |
| | Synthesis-Dependent Strand Annealing | | | |
| | (SDSA) | | | |
| Gene | RNA Polymerase III Abortive And Retractive | −0.0114 | −0.0022 | fALS |
| expression | Initiation | | | down |
| (Transcription) | | | | |
| Gene | RNA Polymerase III Chain Elongation | −0.014 | −0.0028 | fALS |
| expression | | | | down |
| (Transcription) | | | | |
| Gene | RNA Polymerase III Transcription Initiation | −0.0109 | −0.0013 | fALS |
| expression | From Type 3 Promoter | | | down |
| (Transcription) | | | | |
| Gene | RNA Polymerase III Transcription | −0.0129 | −0.0026 | fALS |
| expression | Termination | | | down |
| (Transcription) | | | | |
| Gene | RUNX3 regulates p14-ARF | 0.117 | 0.0545 | fALS up, |
| expression | | | | sALS up |
| (Transcription) | | | | |
| Gene | RUNX3 regulates WNT signaling | −0.0119 | 0 | fALS |
| expression | | | | down |
| (Transcription) | | | | |
| Gene | TFAP2 (AP-2) family regulates transcription | −0.0028 | −0.0713 | sALS |
| expression | of growth factors and their receptors | | | down |
| (Transcription) | | | | |
| Gene | Transcriptional regulation by RUNX3 | 0.0404 | 0.0191 | fALS up, |
| expression | | | | sALS up |
| (Transcription) | | | | |
| Metabolism | CYP2E1 reactions | −0.0109 | −0.0047 | fALS |
| | | | | down |
| Metabolism of | Incretin synthesis, secretion, and inactivation | −0.0119 | 0 | fALS |
| proteins | | | | down |
| Metabolism of | Protein methylation | −0.0002 | −0.0121 | sALS |
| proteins | | | | down |
| Metabolism of | Synthesis, secretion, and inactivation of | −0.0119 | 0 | fALS |
| proteins | Glucagon-like Peptide-1 (GLP-1) | | | down |
| Muscle | Phase 2-plateau phase | 0.0001 | 0.0125 | sALS up |
| contraction | | | | |
| Neuronal | Activation of GABAB receptors | −0.0166 | −0.0001 | fALS |
| System | | | | down |
| Neuronal | GABA B receptor activation | −0.015 | −0.0001 | fALS |
| System | | | | down |
| Neuronal | GABA receptor activation | −0.0135 | −0.0001 | fALS |
| System | | | | down |
| Neuronal | Adenylate cyclase inhibitory pathway | −0.0259 | −0.0001 | fALS |
| System, Signal | | | | down |
| Transduction | | | | |
| Programmed | Stimulation of the cell death response by | 0.0017 | 0.0101 | sALS up |
| Cell Death | PAK-2p34 | | | |
| Reproduction | Reproduction | 0.0106 | −0.0006 | fALS up |
| Signal | Binding of TCF-LEF:CTNNB1 to target | −0.0119 | 0 | fALS |
| Transduction | gene promoters | | | down |
| Signal | G-protein activation | −0.0101 | −0.0003 | fALS |
| Transduction | | | | down |
| Signal | G-protein mediated events | −0.0122 | −0.0001 | fALS |
| Transduction | | | | down |
| Signal | Hedgehog ligand biogenesis | 0.0001 | 0.0185 | sALS up |
| Transduction | | | | |
| Transport of | ABC-family proteins mediated transport | 0.0006 | 0.0104 | sALS up |
| small | | | | |
| molecules | | | | |
| Transport of | Bicarbonate transporters | −0.0202 | 0 | fALS |
| small | | | | down |
| molecules | | | | |

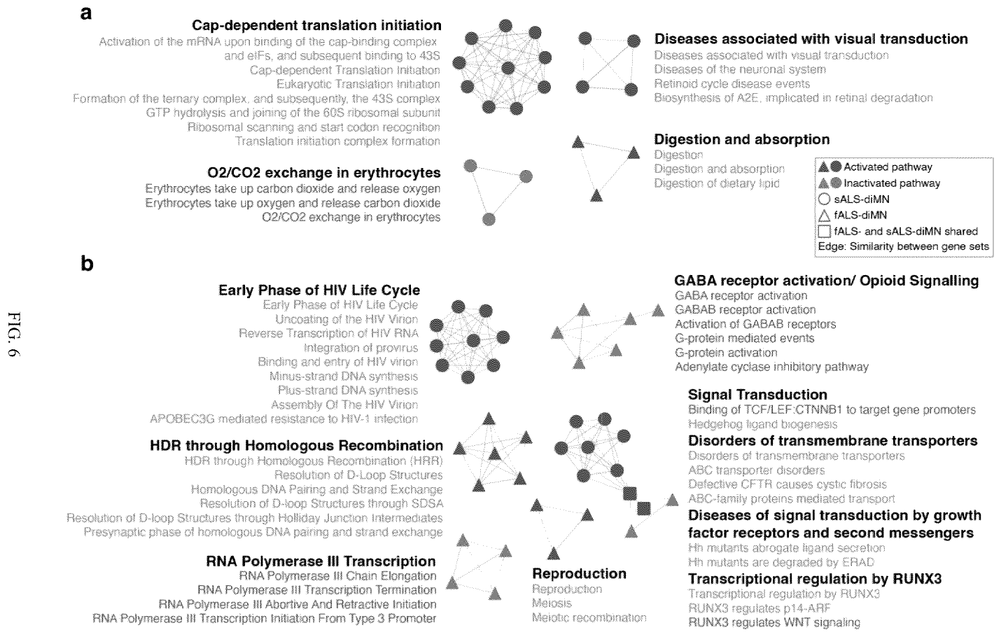

The dysregulated pathways in the diMN ALS groups belonged to different biological processes when compared to the CNS groups. For the diMN transcriptomics comparisons, pathways of oxygen and carbon dioxide exchange were found to be inhibited, and pathways of the cap-dependent translation, disease associated with visual transduction, and digestion and absorption were found to be activated in ALS case groups (FIG. 6a). For the diMN proteomics comparisons, the RNA polymerase III transcription and GABA receptor pathways were found to be inhibited (FIG. 6b). On the other hand, pathways of the early phase of HIV life cycle, homologous recombination of DNA repair, and reproduction were activated. The pathways related to signal transduction and its related diseases, transmembrane transporter disorders, and transcriptional regulation by RUNX3 formed the largest cluster due to their shared genes of the ubiquitin-proteasome system, such as the ubiquitin genes (UBC, UBB and UBA52), the proteasome genes (SEM1, RPS27A, and PSM subunits), and the endoplasmic reticulum (ER)-associated degradation genes (VCP, SEL1L, OS9, ERLEC1, and DERL2).

### Targets Based on CNS Data

Fourteen high confidence, one novel to ALS and eight novel to all disease targets are selected based on CNS data. They are mainly involved in the biological process of apoptosis and inflammation (Table 5).

### CNS Targets and Apoptosis

### MAP3K5

Neuronal cell death serves as the ultimate consequence of various ALS pathogenic signatures, such as mitochondrial dysfunction and excitotoxicity. Apoptosis and necroptosis are the two pivotal mechanisms underlying motor neuron loss. Among the CNS comparisons, multiple targets with altered expression profiles contributed to neuronal apoptosis that might serve as therapeutic targets to tackle neurodegeneration. MAP3K5 regulates the activities of p38 and JNK in response to various environmental stresses. Accumulating evidence indicated the contribution of MAP3K5 activation to neurodegeneration. In ALS, lymphocytes isolated from the patients displayed elevated levels of MAP3K5 when compared with healthy individuals. Activated MAP3K5 was markedly increased in the motor neurons of SOD1 transgenic mice. Our data also showed a general upregulation of MAP3K5 in CNS fALS comparisons (80%). Moreover, several studies revealed the linkage between SOD1 mutant and MAP3K5 activation in neuronal cell death. Administration of MAP3K5 inhibitors prolongs survival of SOD1^{mut} mice through blocking MAP3K5 activity and glial activation in the spinal cord[4], indicating the significance of MAP3K5 upregulation in ALS.

### NOSI

Following p38 activation by MAP3K5, ALS-driven gene transcription stimulates the activities of toxic enzymes, including neuronal nitric oxide synthase (nNOS). nNOS synthesizes nitric oxide (NO) from L-arginine. It is encoded by NOS1, a target that was overexpressed in over 80% of both fALS and sALS CNS comparisons. NO itself is nontoxic and essential for neural communication. However, mitochondrial dysfunction in ALS stimulates the production of a superoxide anion, which immediately reacts with NO to form a potent oxidant-peroxynitrite. Peroxynitrite is neurotoxic in terms of its action on inhibiting mitochondrial proteins, suppressing ATP synthesis, and DNA damage. In addition, increased NO level in cultured motor neuron cells promoted mutant SOD1 aggregation, and this scenario was prevented by the use of NOS signaling inhibitors. Recent studies also illustrated the overexpression of nNOS as a common event in ALS[8], which aligned with our findings. Taking these facts into account, we speculate that together with MAP3K5 overexpression, upregulation of NOS1 amplifies the production of NO and peroxynitrite, making motor neurons more vulnerable to reactive oxygen species-induced apoptosis.

### RARA

In contrast to MAP3K5 and NOS1, RARA was generally downregulated in our CNS sALS comparisons (14.3% upregulated). RARA encodes the nuclear retinoic acid receptors alpha (RARα), a transcriptional activator in the retinoid signaling pathway. RAR functions as a heterodimer by binding with retinoid X receptor (RXR) to activate transcription of their downstream targets, and thus trigger various cellular responses, such as apoptosis, cell differentiation, and embryonic development. Retinoids display critical roles in CNS development via promoting neural patterning, neural differentiation, and motor axon outgrowth[9]. Upon nerve injury, retinoid signaling is activated to promote nerve regeneration, as well as suppress inflammatory cytokine production. Deficiency of RARα was observed in motor neurons from sALS patients, and in lumbar spinal cord tissue of SOD1^{G93A} mice. Goncalves et al. showed that RARαagonist stimulates Aβ clearance and attenuated inflammation in Alzheimer's disease (AD) in vivo, suggesting a potential therapeutic role of RARα as a neuroprotective agent in treating neurodegeneration.

### CNS Targets and Inflammation

### PTPRC

With evidence in both ALS patients and in vivo models, neuroinflammation has posed a detrimental effect on ALS progression. It is marked by microglial activation, infiltration of macrophages, as well as complement signaling activation in the CNS tissue. Several selected candidates (Table 5) were functionally associated with immune regulation, or with evidence participating in the inflammatory response in neurodegeneration. For instance, upregulation of PTPRC is detected in 80% of CNS fALS and 86% of sALS comparisons. PTPRC encodes CD45, a transmembrane protein tyrosine phosphatase expressed in all leukocytes. CD45 takes a key role in orchestrating T cell antigen receptor signaling. Its depletion could result in severe immunodeficiency due to T cell and B cell dysfunction. Data from a gene co-expression network analysis suggests PTPRC acts as a top hub gene in the immune/microglia module in patients with neurodegeneration. It is noteworthy that entry of immune cells into the brain is strictly controlled by the blood-brain barrier, unless there is a breakage in the barrier. It is proposed PTPRC might serve as a potential biomarker for ALS and its expression is positively correlated with inflammatory cell counts in ALS patients[10]. Furthermore, enrichment of PTPRC protein is also reported in AD human tissue and mouse models, indicating the possible connection between PTPRC and neurodegenerative disease.

### NR3C1

Another example is NR3C1, the gene that encodes glucocorticoid receptors. NR3C1 has a dual mode of action by functioning as a modulator of transcription factors and as a transcription factor itself. The expression of NR3C1 in the CNS tissue was found to be generally upregulated in both fALS (80%) and sALS (86%) comparisons. Immunosuppression therapy involving two NR3C1 agonists, viz. methylprednisolone and prednisone, was tested in ALS but none of the patients reached the pre-defined responder criteria after treatment. However, the therapeutic potential of an NR3C1 antagonist (CORT113176) was revealed in the ALS-mimic mouse model by reducing the expression and origin of pro-inflammatory factors. Given the up-regulated expression of NR3C1 in ALS comparisons as well as the supportive preclinical evidence of NR3C1 antagonists, NR3C1 could be a potent actionable candidate for the treatment of ALS patients, especially those with high inflammation markers.

### Novel CNS Targets

### STUB1

Despite the majority of CNS targets underlying neuronal cell death and inflammation, several targets were functionally correlated with the early-stage ALS characters, especially for novel targets (Table 5). STUB1 was consistently downregulated in both CNS fALS (20% upregulated) and sALS (14.3% upregulated) comparisons. It is a well-known protein with dual roles serving as both co-chaperone and E3 ubiquitin ligase. Growing evidence suggests that the impact of STUB1 on protecting neurons against the cellular toxicity of pathogenic protein aggregates. Overexpressing STUB1 reduces cytotoxicity by promoting mutant SOD1 elimination[11]. Dorfin is recognized for its activity in the ubiquitination of mutant SOD1 proteins but has low stability. STUB1 stabilizes Dorfin in the form of chimeric proteins and enhances its efficacy of mutant SOD1 ubiquitination and degradation in vivo. STUB1 also protects against AD and Parkinson's disease (PD) through increasing the degradation of Aβ plaques and α-Synuclein, respectively. These altogether delineate the importance of STUB1 in acting as a safeguard to prevent neurons from mutant SOD1-induced cytotoxicity.

### ATP5F1A

Impaired mitochondrial function is an emerging critical pathophysiological condition that drives neurodegeneration, including ALS. Elevated oxidative stress, reduced ATP production, mitochondrial DNA mutations, and F₁F₀ ATP synthase malfunction collectively contribute to mitochondrial dysfunction. ATP synthase is responsible for the final stage of oxidative phosphorylation. The imbalance of the F₁/F₀ ratio in ATP synthase is indicated as a disease driving force in AD and PD. ATP5F1A, encoding F₁ subunit alpha, was downregulated in all CNS sALS comparisons. Aligning with our finding, downregulation of ATP5F1A is reported in brain tissue of patients with C9orf72-mediated ALS, and as a consequence of protein ubiquitination induced by poly (GR) peptides[12]. Although limited knowledge is available regarding the relationship between ATP5F1A and ALS, it is worth further examination due to the growing attention on how ATP synthase works in fighting ALS.

TABLE 5
List of therapeutic targets for amyotrophic lateral sclerosis based on CNS data

| | | | | | | | |
|---|---|---|---|---|---|---|---|
| Gene¹ | fALS² | sALS² | Proposed therapy³ | Protein family | Tissue enrichment⁴ | Proposed ALS mechanism | # Trials |

High confidence

| | | | | | | | |
|---|---|---|---|---|---|---|---|
| ADRA2B | 80% | 50% | Antagonist (fALS) | GPCR | Low tissue specificity | Protein degradation | 1,577 |
| CYBB | 80% | 86% | Antagonist | Ion channel | Blood, lung, lymphoid tissue | Oxidative stress | 0 |
| FLT1* | 80% | 57% | Antagonist (fALS) | Receptor kinase | Placenta | Inflammation | 3,154 |
| HDAC1* | 80% | 43% | Antagonist (fALS) | Hydrolase | Low tissue specificity | Apoptosis | 1,302 |
| IGF1R* | 80% | 43% | Antagonist (fALS) | Receptor kinase | Low tissue specificity | Apoptosis | 269 |
| MAP3K5* | 80% | 71% | Antagonist (fALS) | Protein kinase | Adrenal gland | Apoptosis | 6 |
| MAPK1* | 80% | 71% | Antagonist (fALS) | CMGC kinase | Brain | Apoptosis | 12 |
| MLKL | 100% | 43% | Antagonist (fALS) | Tyrosine kinase-like | Vagina | Apoptosis | 0 |
| NOS1 | 80% | 86% | Antagonist | Oxidoreductase | Brain and skeletal muscle | Oxidative stress | 58 |
| NR3C1* | 80% | 86% | Antagonist | Nuclear receptor | Low tissue specificity | Inflammation | 7,761 |
| PTK2 | 40% | 86% | Antagonist (sALS) | Tyrosine kinase | Low tissue specificity | Protein aggregation | 45 |
| PTPRC | 80% | 86% | Antagonist | Receptor phosphatase | Blood, lymphoid tissue | Inflammation | 9 |
| RARA | 50% | 14% | Agonist (sALS) | Nuclear receptor | Low tissue specificity | Neurogenesis | 500 |
| VDR | 60% | 14% | Agonist (sALS) | Nuclear receptor | Intestine, parathyroid gland | Apoptosis | 1,531 |

Novel to ALS

| | | | | | | | |
|---|---|---|---|---|---|---|---|
| KCNB2 | 60% | 83% | Antagonist (sALS) | Ion channel | Brain, lymphoid tissue, pituitary | Excitotoxicity | 64 |
| | | | | | gland | | |

Novel to all diseases

| | | | | | | | |
|---|---|---|---|---|---|---|---|
| AHCYL1 | 100% | 71% | Antagonist | Enzyme | Low tissue specificity | Apoptosis | 0 |
| ATP5F1A | 50% | 0% | Agonist (sALS) | Hydrolase | Low tissue specificity | Mitochondrial dysfunction | 0 |
| NR2F6 | 60% | 14% | Agonist (sALS) | Nuclear receptor | Low tissue specificity | Inflammation | 0 |
| P2RY14 | 40% | 14% | Agonist (sALS) | GPCR | Granulocytes, dendritic cells, | Inflammation | 0 |
| | | | | | placenta | | |
| PDIA6 | 60% | 71% | Antagonist (sALS) | Isomerase | Low tissue specificity | Protein aggregation | 0 |
| SCYL1* | 40% | 14% | Agonist (sALS) | Protein kinase | Low tissue specificity | Apoptosis | 0 |
| SLC25A10 | 20% | 29% | Agonist | Transporter | Liver | Oxidative stress | 0 |
| STUB1* | 20% | 14% | Agonist | Acyltransferase | Low tissue specificity | Protein degradation | 0 |

¹Manually curated aging-associated genes (marked with *) based on pathways suggested by López-Otín et al. or GenAge database (https://genomics.senescence.info/genes/index.html);
²Percentage of comparisons with up-regulated target (LFC > 0) out of five fALS or seven sALS comparisons;
³Therapy proposed based on expression alterations in fALS and/ or sALS comparisons;
⁴Tissue enrichment (RNA) retrieved from Human Protein Atlas (https://www.proteinatlas.org/).

Targets Based on diMN Data

Seven high confidence, two novel to ALS, and three novel to all disease targets are selected based on diMN data (Table 6)

diMN Targets and Proteostasis Disturbance

### ERN1

Accumulation of misfolded proteins, i.e., SOD1 and TDP-43 aggregates, has long been demonstrated as a cause of ALS. Several dysregulated targets identified from diMN comparisons (Table 6) were likely to be associated with impaired proteostasis in ALS and other neurodegenerative diseases, such as ERN1 and PPP3CB. The unfolded protein response (UPR) serves as a critical stress response to cope with ER stress and maintain cell viability. IRE1 is one of the primary sensors for UPR. The mRNA level of ERN1, encoding IRE1, is found to be upregulated in diMN fALS samples (LFC=0.2058, p=0.003). IREI signaling is considered to be pathogenic in AD and PD. The administration of PPAR agonist exerts its protective effect on neurodegeneration through suppression of IRE1-mediated ER stress response. In SOD1^{G93A} mice, IRE1 protein level is elevated with disease progression[13]. Downstream targets of the IRE1 pathway are also reported to be activated in the spinal cord of ALS patients. These altogether confer the therapeutic possibility of targeting ERN1 in ALS.

### PPP3CB

Calcineurin (Cn) is a calcium-and calmodulin-dependent serine/threonine phosphatase that participates in Ca²⁺-dependent signaling transduction to dephosphorylate and activate multiple proteins, such as SSH1, DNM1L, and NFAT. PPP3CB encodes the β-isoform of its catalytic subunit. It has been reported that the activity of Cn is correlated with SOD1 and TDP-43. SOD1 serves as the upstream regulator of Cn and stabilizes its activity via active site interaction. Alteration in the conformation state of SOD1 impairs its interaction with Cn. As a consequence, the weakening of SOD1^{G93A}-Cn interaction in SOD^{G93A} mice decreased Cn stability, leading to the defect in TDP-43 dephosphorylation. TDP-43 aggregation—one of the critical pathogenic features in ALS—was therefore detected in the spinal cord tissue[14]. Moreover, trehalose, a natural compound tested to be beneficial in multiple neurodegenerative models, executes its function via activating PPP3CB/Cn. PPP3CB stimulates the activity of transcription factor EB, and eventually promotes autophagy to ameliorate neurodegeneration. Our result further supported these findings by indicating the reduction of PPP3CB protein level in diMN fALS samples (LFC=−0.3048, p=0.0115).

diMN Targets and Compromised Mitochondrial Function

### VCP

Cancer, neurodegenerative and cardiovascular diseases are documented as the manifestations of redox imbalance. Mounting evidence indicates the implication of oxidative stress in promoting neurodegeneration. VCP is dysregulated in diMN proteomics comparisons (Table 6). VCP has been evaluated as an ALS-linked gene in the recent decade, and its mutations are reported in 1-2% of ALS patients. Positive correlation is observed between VCP-positive cell count and severity of the disease in ALS patients. Functionally, VCP mutations are closely linked with protein mislocalization, as well as deterioration of mitochondrial function. Knock-in mouse model with VCPR155H mutation resembles the key feature of ALS, including pronounced motor neuron loss, TDP-43 aggregation, and mitochondrial malfunction. As a result, VCP inhibitor has drawn attention due to its favourable outcome on relieving ALS phenotypes. Recently, pharmacological inhibition of VCP effectively corrects mislocalization of RNA-binding proteins, including SOD1 and TDP-43, in VCP mutant motor neurons[15]. Here our finding was aligned with the literature by showing upregulation of VCP in sALS samples (LFC=0.0776, p=0.0411).

### G6PD

G6PD, the key cytosolic enzyme responsible for the production of antioxidant NADPH, was also found to be dysregulated in fALS samples (LFC=−0.1416, p=0.0487). Babu et al. reported progressive reduction in G6PD activity during disease development in sALS patients, which is in accordance with our result. NADPH is an essential reducing agent in all organisms. Depletion of NADPH triggers oxidative stress and subsequently induces DNA damage and cell death. In aged mice, increase in G6PD level offers a protective effect against oxidative stress-induced neurodegeneration[16]. Similar results are also observed in an in vivo PD model. Treatment with G6PD agonist restores redox balance in zebrafish and in vitro models. Although it is debatable whether targeting mitochondrial dysfunction is efficient in treating neurodegeneration, these findings enrich our knowledge on the correlation between mitochondrial homeostasis and ALS.

TABLE 6
List of therapeutic targets for amyotrophic lateral sclerosis based on diMN data

| | | | | | | | |
|---|---|---|---|---|---|---|---|
| | | | Proposed | | | Proposed ALS | |
| Gene¹ | fALS LFC (p)² | sALS LFC (p)² | therapy³ | Protein family | Tissue enrichment⁴ | mechanism | # Trials |

High confidence

| | | | | | | | |
|---|---|---|---|---|---|---|---|
| PPIA | 0.1728 (0.0338) | 0.2361 (0.0003) | Antagonist (P) | Isomerase | Low tissue specificity | TDP-43 pathology, | 1,376 |
| | | | | | | (inflammation) | |
| DNMT3A | 0.1324 (0.0346) | 0.0773 (0.1172) | Antagonist (T) | Methyltransferase | Low tissue specificity | Apoptosis | 1,038 |
| ERN1 | 0.2058 (0.003) | 0.0699 (0.1644) | Antagonist (T) | Protein kinase | Low tissue specificity | Protein aggregation | 0 |
| HSPD1* | 0.1363 (0.1365) | 0.1916 (0.0083) | Antagonist (P) | Isomerase | Vagina | FUS pathology, | 0 |
| | | | | | | inflammation | |
| RPS6KB1 | 0.1558 (0.0297) | 0.1426 (0.0052) | Antagonist (T) | AGC kinase | Low tissue specificity | Protein aggregation | 12 |
| VCP | 0.0212 (0.637) | 0.0776 (0.0411) | Antagonist (P) | Hydrolase | Low tissue specificity | Mitochondrial | 0 |
| | | | | | | dysfunction | |
| G6PD | −0.1416 (0.0487) | −0.0847 (0.1337) | Agonist (P) | Oxidoreductase | Testis | Oxidative stress | 0 |

Novel to ALS

| | | | | | | | |
|---|---|---|---|---|---|---|---|
| KCNS3 | 0.3886 (0.0995) | 0.3338 (0.0282) | Antagonist (T) | Ion channel | Skeletal muscle | Excitotoxicity | 64 |
| PSMC6* | −0.2636 (0.0402) | −0.1502 (0.0926) | Agonist (P) | Hydrolase | Low tissue specificity | Proteostasis | 3 |

Novel to all diseases

| | | | | | | | |
|---|---|---|---|---|---|---|---|
| METTL21A | 0.196 (0.0012) | 0.0827 (0.0432) | Antagonist (T) | Methyltransferase | Low tissue specificity | Protein aggregation | 0 |
| TOPORS | 0.2161 (0.0135) | 0.1385 (0.0184) | Antagonist (T) | Acyltransferase | Low tissue specificity | Apoptosis | 0 |
| PPP3CB* | −0.3048 (0.0115) | −0.1371 (0.1725) | Agonist (P) | Esterase | Skeletal muscle | Protein aggregate | 0 |
| | | | | | | degradation | |

¹Manually curated aging-associated genes (marked with *) based on pathways suggested by López-Otín et al. or GenAge database (https://genomics.senescence.info/genes/index.html);
²The therapy was proposed for the underscored subtype(s) based on both expression alteration and TargetID ranking;
³Therapy proposed based on expression alterations using transcriptomics (T) or proteomics (P) data;
⁴Tissue enrichment (RNA) retrieved from Human Protein Atlas (https://www.proteinatlas.org/).

### Potential Repurposing Candidates

With the proposed therapies for each target (Tables 5 and 6), drugs tackling our target genes were curated and evaluated for their safety, mechanism of action (MOA), and blood-brain barrier penetrability. The refined list of repurposing candidates includes thirteen drugs targeting nine different genes (Table 7). All the proposed candidates have been investigated in one or more nervous system disorders with clinical trials ranging from phase 2 to 4 (launched).

### Mirtazapine

Mirtazapine functions as a strong antagonist of serotonin receptors and adrenergic receptors, including ADRA2B, to stimulate norepinephrine and serotonin secretion in patients suffering from depression. In current study, ADRA2B was upregulated in 80% of CNS fALS comparisons. Treatment of ADRA2 agonist aggravates disease phenotypes in SOD1^{G93A} mice. In consideration of the above facts, mirtazapine is regarded as one of the repurposing candidates for ALS treatment.

### Dalfampridine

Dalfampridine is a potassium ion channel blocker approved to facilitate the mobility of patients with multiple sclerosis. A few studies report KCNS3 as a risk gene in AD and PD. However, there is no direct evidence of the association of potassium ion channel expression upregulation and ALS. Since upregulations of KCNB2 and KCNS3 were detected in our study, dalfampridine could be a potential therapeutic for ALS in light of its good efficacy and acceptable safety profile in treating patients with multiple sclerosis.

### Acitretin

Acitretin, an agonist of retinoic acid receptors, has been approved to treat severe psoriasis. Besides, it is under investigation for AD in terms of its anti-amyloidogenic and immune-stimulatory effects. According to the general downregulation in CNS ALS comparisons and the neuroprotective role of RARA, acitretin was proposed for repurposing to treat ALS. Given the adverse effects of causing birth defects, acitretin therefore might be contraindicated in female patients who are pregnant or intend to become pregnant.

TABLE 7
List of repurposing candidates for amyotrophic lateral sclerosis.

| | | Max | | Target | BBB | | Natural | Associated neurological |
|---|---|---|---|---|---|---|---|---|
| Drug | Target | phase¹ | Drug MOA | genes² | penetration³ | IC50⁴ | compound⁵ | diseases⁶ |
| Mirtazapine | ADRA2B | Launched | Antagonist of | HTR2A, | 0.9855 | — | — | Depression [NCT00782405], |
| | | | ADRA2B, | ADRA2B, | | | | Cocaine dependence |
| | | | HTR2C | ADRA2A, | | | | [NCT00249444], |
| | | | and HTR2A | HTR2C, | | | | Alcohol dependence |
| | | | | OPRK1, | | | | [NCT00874003], etc. |
| | | | | HRH1 | | | | |
| Labetalol | ADRA2B | Launched | ADRA2B | ADRB1, | 0.8313 | 13.2 mg/L in | — | Stroke [NCT02327793], |
| | | | antagonist | ADRB2 | | human | | Nicotine dependence |
| | | | | | | neutrophils | | [NCT00000297], Cocaine |
| | | | | | | | | dependence [NCT00000291], |
| | | | | | | | | Intracerebral hemorrhage |
| | | | | | | | | [NCT00963976] |
| Azacitidine | DNMT3A | Launched | Pyrimidine | DNMT1, | 0.8753 | 0.019 μg/mL | — | CNS cancer [NCT03666559], |
| | | | nucleoside | DNMT3A | | on mouse | | childhood ependymoma |
| | | | analogue | | | leukemia | | [NCT03206021], |
| | | | | | | cells | | ependymoma |
| | | | | | | | | [NCT03572530], etc |
| Decitabine | DNMT3A | Launched | Pyrimidine | DNMT1, | 0.8787 | 0.1 μM on | — | Medulloblastoma |
| | | | nucleoside | DNMT3A, | | leukemia | | [NCT02332889], |
| | | | analogue | DNMT3B | | cells | | neuroblastoma |
| | | | | | | at 24 h | | [NCT00075634] |
| AXL-1717 | IGF1R | P2 | IGF1R | IGF1R | — | 1 nM for | — | Recurrent malignant |
| | | | inhibitor | | | IGF1R | | astrocytomas |
| | | | | | | | | [NCT01721577] |
| Dalfampridine | KCNB2 | Launched | Voltage-gated | KCNA, | 0.9544 | 290 μM for | NPC57565 | Multiple sclerosis |
| | | | potassium | KCNB, | | KCNA1 | | [NCT01480076], Stroke |
| | | | channel | KCNC, | | 1.5 mM | | [NCT02422940], Spinal cord |
| | | | blocker | KCND | | for KCNB2 | | injury [NCT00041717], etc. |
| | | | | subfamilies | | 29 μM for | | |
| | | | | | | KCNC1 | | |
| Ulixertinib | MAPK1 | P2 | MAPK1 | MAP3, | — | <0.3 nM for | — | Uveal melanoma |
| | | | inhibitor | MAPK1 | | ERK2* | | [NCT03417739] |
| Ronopterin | NOS1 | P3 | NOS inhibitor | NOS | — | — | — | Traumatic brain injury |
| Mifepristone | NR3C1 | Launched | Antagonist of | PGR, | 0.7135 | 0.2 nM for | SN00064357 | Depression [NCT00128479], |
| | | | NR3C1 and | NR3C1, | | NR3C3* | | Alcohol dependence |
| | | | NR3C3 | KLK3, | | 2.6 nM for | | [NCT01548417], |
| | | | | NR1I2 | | NR3C1* | | Bipolar disorder |
| | | | | | | | | [NCT00043654], |
| | | | | | | | | Meningioma |
| | | | | | | | | [NCT03015701], etc. |
| ORG-34517 | NR3C1 | P2 | NR3C1 | NR3C1 | — | 17.9 nM for | — | Depression [NCT00212797] |
| | | | antagonist | | | NR3C1* | | |
| Cyclosporine | PPIA | Launched | Calcineurin | PPIA | — | 7 nM for | — | Juvenile dermatomyositis |
| | | | inhibitor | | | calcineurin | | [NCT00323960], |
| | | | | | | | | neuroblastoma |
| | | | | | | | | [NCT00874315], |
| | | | | | | | | retinoblastoma |
| | | | | | | | | [NCT00110110], etc |
| Acitretin | RARA | Launched | Retinoid | RXRA, | 10.5841 | 6.6 μM for | — | Alzheimer's disease |
| | | | receptor agonist | RARA, | | proliferation | | [NCT01078168] |
| | | | | RARB, | | | | |
| | | | | RARG, | | | | |
| | | | | RXRB, | | | | |
| | | | | RXRG, | | | | |
| | | | | RBP1 | | | | |
| Tamibarotene | RARA | P2 | RAR-α agonist | RARA, | 0.9589 | 6.9 nM for | — | Alzheimer's disease |
| | | | | RARB | | RARA | | [NCT01120002] |

¹Maximum phase in ClinicalTrials.gov;
²Retrieved from DrugBank or ChEMBL;
³Predicted probability in DrugBank or inferred based on the disease type the drug was tested in;
⁴Retrieved from Selleckchem.com (marked by *) or other websites;
⁵Match of drug name in Super Natural II or NPASS (NPC) database;
⁶ID of the maximum-phased clinical trial of the disease shown in brackets.

### 6. Discussion

After decades of research, the involvements of multiple factors and pathogenic variations among ALS patients make it difficult to draw a conclusive pathophysiologic process of ALS. In order to obtain a better understanding of the biology of ALS, integrative multi-omics approaches were applied to dissect the disease physiology. PandaOmics™ is a fully integrated AI-based platform with a wide range of omics and text data sources. Compared to other existing tools for target discovery, PandaOmics™ has several unique advantages with respect to user experience, algorithms, the comprehensive database, and the time machine validation approach. The platform not only offers differential gene expression and pathway analysis but also a ranking of target genes based on a dynamic calculation of the selected datasets, either custom uploaded or pre-processed, in PandaOmics™. More importantly, multi-layer validations were included to estimate each model's performance with respect to the type of experimental data, protein family, disease area, and ability to suggest novel targets, etc. The time machine validation approach clearly demonstrated the power of the platform to find novel targets. With just a few clicks, the platform is able to propose druggable targets using multiple advanced bioinformatics and AI models, accelerating the drug discovery process. Therefore, PandaOmics™ is a unique and user-friendly AI-driven target discovery platform for therapeutic target exploration based on multi-omics data analysis that requires no prior knowledge of computational biology.

With the advance of medical care and improved lifestyles, human life expectancy has been significantly lengthened, which in turn poses significant health-associated challenges in our society due to the shift in demographic structure toward the aged. A wide variety of disorders are proven to be related to biological aging. Despite multiple risk factors being proposed to contribute to ALS, aging remains as one of the most prevalent risk factors[17]. Various molecular and cellular mechanisms, for instance, telomere attrition, mitochondrial dysfunction, and cell senescence contribute to the process of aging. Among our shortlisted therapeutic targets, several are suggested to be aging-associated, such as IGF1R, HDAC1,and PPP3CB. However, the mechanisms of how these targets function during aging remain controversial. For example, IGF1R signaling is beneficial to cognitive and cardiac health in aged rodent models. A population-specific gene expression analysis revealed that IGF1R expression decreased with age in PBMC extracted from Polish Caucasians, and proposed that the decline in IGF1R contributes to pro-inflammatory response. Oppositely, targeting IGF1R by monoclonal antibody expands the lifespan of female mice by 9%, as well as suppresses inflammation and tumorigenesis. Zarse et al. supported this hypothesis by demonstrating that impaired IGF-1 signaling triggers non-glucose metabolism to increase life expectancy.

In the present study, we show that several enriched pathway clusters are closely linked with ALS-driven mechanisms. For example, RNA metabolism was commonly dysregulated in our analysis regardless of tissue type. It is clear that altered RNA metabolism is a key concern in ALS. TDP-43 and FUS are RNA-binding proteins that exert broad impact on RNA metabolism pathways. Mutations in these two genes were proven to induce pathogenic RNA metabolic changes in ALS, such as mRNA translation defect, altered splicing function, and deregulated nonsense-mediated mRNA decay. Independent studies also evaluated the relevance of other ALS genes to RNA metabolism and uncovered that mutant C9orf72 might induce RNA toxicity. In addition, pathways controlling innate immune response and programmed cell death were found to be upregulated in CNS comparisons, in which both neuronal cell death and neuroinflammation are the well-characterized processes in promoting neurodegeneration. The hemostasis and erythropoietin signaling pathways were activated in CNS comparisons, suggesting activated neuro-immune hemostasis network in response to the CNS tissue damage. Excitotoxicity, a pathophysiological condition caused by excessive glutamate stimulation, is suspected as a mediator driving ALS development. This hypothesis is further supported by the action of riluzole, an FDA-approved anti-excitotoxic therapy, in ALS treatment. We also show that GABAergic signaling pathways were downregulated, leading to an increase in glutamate toxicity. It functions to counteract excessive neuronal excitability and offers a calming effect.

It is not surprising that fewer pathways will be uniformly altered in sALS relative to fALS comparisons given the complex genetic bases and the large variations among sporadic ALS individuals (FIG. 1). However, there are some pathway clusters that are specific to sALS, such as the FGFR signaling pathways. Fibroblast growth factors and their receptors play essential roles in the development, maintenance and repair of the nervous system. In adult mammals, FGF signaling is found to increase neuronal survival after spinal cord injury, and support the proliferation of neuronal progenitor cells. The inhibition of FGFR signaling indicates the reduction of neurogenetic effects underlying ALS etiology, which was confirmed in the CNS sALS groups (FIG. 1). However, it was not observed in the CNS fALS groups, which might stem from the lack of association between FGF signaling and C9orf72 mutations that represent the dominant genotype in the fALS comparisons (Table 1).

As the final phase for neurodegeneration, neuronal cell death could be the consequence of apoptosis or necroptosis, an alternative programmed cell death event characterized by inflammation. Neuroinflammation itself could also be a late-stage phenotype in ALS, supported by evidence in both human tissue and animal models[18]. In spite of the fact that both CNS and diMN target lists contain apoptosis-or inflammation-associated genes, we observed the majority of CNS targets (i.e. 61%) contribute to the late-stage signatures of ALS. This observation is in agreement with results obtained from pathway clustering analysis of CNS comparisons, partially due to the impact of late-stage pathological changes in the post-mortem tissues. Furthermore, miscellaneous cell types in CNS samples, consisting of neurons, glial cells, as well as CNS-resident and infiltrated immune cells upon neuronal injury, contribute to the expression profile observed in the analysis. Conversely, targets identified in diMN comparisons are dominantly involved in impaired proteostasis, one of the most well-studied pathophysiologies of early ALS[19]. Unlike CNS samples, transcriptomics and proteomics profiles in diMN ALS samples are solely derived from motor neurons, without the influence of non-neuron cells. Such comparisons could clearly reflect the disease pathology in motor neurons. In the absence of the aging process, it is reasonable that the roles of diMN targets are mainly involved in the early-stage signature of ALS. A recent study for Alzheimer's progression in the human brain highlighted the importance of integrating human data with cell lines and animal models data for a better understanding of different stages of disease development[20]. Therefore, our combinational usage of post-mortem CNS tissue and diMN samples could generate a comprehensive view of ALS pathogenesis.

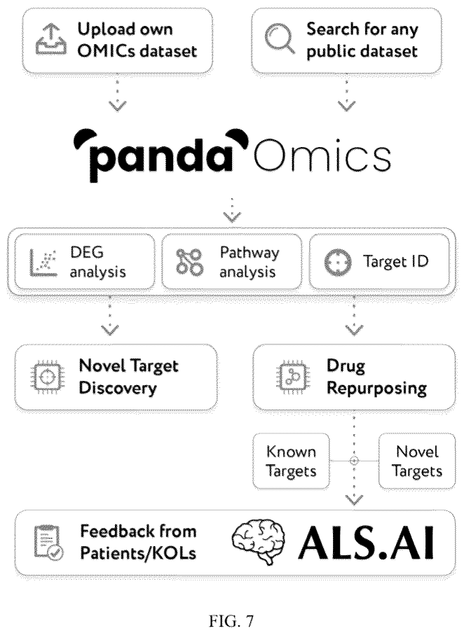

To facilitate communication and collaboration of ALS research community and patients, we have constructed ALS.AI (http://als.ai/), an online interface for novel target disclosure and drug feedback collection for ALS. As is illustrated in FIG. 7, public or personalized datasets will be firstly analyzed in PandaOmics™ for target identification, followed by the curation and releasing of drugs associated with the identified novel and known targets onto ALS.AI. Feedback on the safety and efficacy of proposed drugs will be collected from ALS patients and KOLs, and the best candidates could be selected for further validation.

The current study has a limited number of fALS samples in both post-mortem and diMN comparisons due to the rarity of fALS incidence. Another limitation is the under-representation of racial groups other than the Caucasians in the present analysis. Future studies should include samples from a wider range of populations. ALS is a progressive disorder contributed by numerous interconnected mechanisms. It would be ideal to assess the pathogenic mechanisms underlying every stage of disease development. Yet data analyzed in the current analysis is likely to represent two stages in ALS—the late-stage in postmortem CNS tissue, and the early-stage reflected in diMN samples, harvested on Day 32. This time frame is generally adopted as the maturation time point of diMNs; therefore, this study model might not well-represent the aging effect in ALS disease progression. Including additional diMN differentiation time points might enhance the comprehensiveness of the analysis.

### 7. Conclusion

In the present study, we demonstrated the power of PandaOmics™ to find high confidence and novel targets for ALS with our latest AI models based on comprehensive omics data analysis. Several well-characterized mechanisms in ALS pathology are found to be dysregulated, including the immune system, RNA metabolism, excitotoxicity, as well as programmed cell death. Twenty-three and twelve targets are proposed according to the meta-analyses of CNS and diMN data respectively. Targets identified from CNS data mainly contribute to neuronal cell death and inflammation, which are recognized as late-stage signatures of ALS. In contrast, more diMN targets are attributed to the early-stage signatures. Combining the usage of diMN and post-mortem CNS samples could provide an in-depth understanding of ALS disease progression. To accelerate novel target discovery and drug investigation for ALS, targets identified in this study will be disclosed on ALS.AI.

### 8. References

1. Longinetti, E. and F. Fang, Epidemiology of amyotrophic lateral sclerosis: an update of recent literature. Curr Opin Neurol, 2019. 32(5): p. 771-776.
2. Zou, Z. Y., et al., Genetic epidemiology of amyotrophic lateral sclerosis: a systematic review and meta-analysis. J Neurol Neurosurg Psychiatry, 2017. 88(7): p. 540-549.
3. Doble, A., The pharmacology and mechanism of action of riluzole. Neurology, 1996. 47(6 Suppl 4): p. S233-41.
4. Fujisawa, T., et al., The ASK1-specific inhibitors K811 and K812 prolong survival in a mouse model of amyotrophic lateral sclerosis. Hum Mol Genet, 2016. 25(2): p. 245-53.
5. Rothstein, J., et al., Answer ALS: A large-scale resource for sporadic and familial ALS combining clinical data with multi-omics data from induced pluripotent cell lines. Preprint at https://www.researchsquare.com/article/rs-96858/v1 (2020).
6. Ozerov, I. V., et al., In silico Pathway Activation Network Decomposition Analysis (iPANDA) as a method for biomarker development. Nat Commun, 2016. 7: p. 13427.
7. Sidiropoulos, K., et al., Reactome enhanced pathway visualization. Bioinformatics, 2017. 33(21): p. 3461-3467.
8 Sasaki, S., N. Shibata, and M. Iwata, Neuronal nitric oxide synthase immunoreactivity in the spinal cord in amyotrophic lateral sclerosis. Acta Neuropathol, 2001. 101(4): p. 351-7.
9 Maden, M., Retinoid signalling in the development of the central nervous system. Nat Rev Neurosci, 2002. 3(11): p. 843-53.
10. Salter, M., et al., Initial identification of a blood-based chromosome conformation signature for aiding in the diagnosis of amyotrophic lateral sclerosis. EBioMedicine, 2018. 33: p. 169-184.
11. Urushitani, M., et al., CHIP promotes proteasomal degradation of familial ALS-linked mutant SOD1 by ubiquitinating Hsp/Hsc70. J Neurochem, 2004. 90(1): p. 231-44.
12. Choi, S. Y., et al., C9ORF72-ALS/FTD-associated poly (GR) binds Atp5a1 and compromises mitochondrial function in vivo. Nat Neurosci, 2019. 22(6): p. 851-862.
13. Chen, D., Y. Wang, and E. R. Chin, Activation of the endoplasmic reticulum stress response in skeletal muscle of G93A*SOD1 amyotrophic lateral sclerosis mice. Front Cell Neurosci, 2015. 9: p. 170.
14. Kim, J. M., et al., Impaired Cu—Zn Superoxide Dismutase (SOD1) and Calcineurin (Cn) interaction in ALS: a presumed consequence for TDP-43 and zinc aggregation in Tg SOD1(G93A) rodent spinal cord tissue. Neurochem Res, 2019. 44(1): p. 228-233.
15. Harley, J., et al., TDP-43 and FUS mislocalization in VCP mutant motor neurons is reversed by pharmacological inhibition of the VCP D2 ATPase domain. Brain Commun, 2021. 3(3): p. fcab 166.
16. Jeng, W., M. M. Loniewska, and P. G. Wells, Brain glucose-6-phosphate dehydrogenase protects against endogenous oxidative DNA damage and neurodegeneration in aged mice. ACS Chem Neurosci, 2013. 4(7): p. 1123-32.
17. Pandya, V. A. and R. Patani, Decoding the relationship between ageing and amyotrophic lateral sclerosis: a cellular perspective. Brain, 2020. 143(4): p. 1057-1072.
18. Fiala, M., et al., IL-17A is increased in the serum and in spinal cord CD8 and mast cells of ALS patients. J Neuroinflammation, 2010. 7: p. 76.
19. Medinas, D. B., V. Valenzuela, and C. Hetz, Proteostasis disturbance in amyotrophic lateral sclerosis. Hum Mol Genet, 2017. 26(R2): p. R91-R104.
20 Penney, J., W. T. Ralvenius, and L. H. Tsai, Modeling Alzheimer's disease with iPSC-derived brain cells. Mol Psychiatry, 2020. 25(1): p. 148-167.

1. A method for treating amyotrophic lateral sclerosis (ALS) in a subject in need thereof, comprising administering to the subject an effective amount of one or more active agents selected from the group consisting of a retinoic acid receptor alpha (RARα) agonist, a voltage-gated potassium channel (KCNB2) inhibitor, an adrenergic receptor α2B (ADRA2B) antagonist, a DNA methyltransferase 3 alpha (DNMT3A) antagonist, an insulin like growth factor 1 receptor (IGF1R) inhibitor, a mitogen-activated protein kinase 1 (MAPK1) inhibitor, a nitric oxide synthase 1 (NOS1) inhibitor, a glucocorticoid receptors (NR3C1) antagonist, and a peptidylprolyl Isomerase A (PPIA) antagonist.

2. The method according to claim 1, wherein:
the adrenergic receptor α2B (ADRA2B) antagonist is selected from mirtazapine and derivative thereof;
the retinoic acid receptor alpha (RARα) agonist is selected from acitretin and derivative thereof;
the voltage-gated potassium channel (KCNB2) inhibitor is selected from dalfampridine and derivative thereof:
the DNA methyltransferase 3 alpha (DNMT3A) antagonist is selected from azacitidine, decitabine and derivatives thereof;
the insulin like growth factor 1 receptor (IGF1R) inhibitor is selected from AXL-1717 and derivative thereof;
the mitogen-activated protein kinase 1 (MAPK1) inhibitor is selected from ulixertinib and derivative thereof;
the nitric oxide synthase 1 (NOS1) inhibitor is selected from ronopterin and derivative thereof;
the glucocorticoid receptors (NR3C1) antagonist is selected from mifepristones, ORG-34517 and derivatives thereof; and/or
the peptidylprolyl Isomerase A (PPIA) antagonist is selected from cyclosporine and derivative thereof.

3-10. (canceled)

11. The method according to claim 1, comprising administering to the subject an effective amount of a first active agent and a second active agent, wherein the first active agent and the second active agent are different from each other and are independently selected from the group consisting of the retinoic acid receptor alpha (RARα) agonist, the voltage-gated potassium channel (KCNB2) inhibitor, the adrenergic receptor α2B (ADRA2B) antagonist, the DNA methyltransferase 3 alpha (DNMT3A) antagonist, the insulin like growth factor 1 receptor (IGF1R) inhibitor, the mitogen-activated protein kinase 1 (MAPK1) inhibitor, the nitric oxide synthase 1 (NOS1) inhibitor, the glucocorticoid receptors (NR3C1) antagonist and the peptidylprolyl Isomerase A (PPIA) antagonist.

12. The method according to claim 11, wherein:
the first active agent is the retinoic acid receptor alpha (RARα) agonist and the second active agent is the adrenergic receptor α2B (ADRA2B) antagonist;
the first active agent is the voltage-gated potassium channel (KCNB2) inhibitor and the second active agent is the adrenergic receptor α2B (ADRA2B) antagonist; or
the first active agent is the retinoic acid receptor alpha (RARα) agonist and the second active agent is the voltage-gated potassium channel (KCNB2) inhibitor.

13-14. (canceled)

15. The method according to claim 11, wherein the first active agent and the second active agent are administered simultaneously, separately or sequentially.

16. The method according to claim 1, comprising administering to the subject an effective amount of a first active agent, a second active agent and a third active agent, wherein the first active agent, the second active agent and the third active agent are different from each other and are independently selected from the group consisting of the retinoic acid receptor alpha (RARα) agonist, the voltage-gated potassium channel (KCNB2) inhibitor, the adrenergic receptor α2B (ADRA2B) antagonist, the DNA methyltransferase 3 alpha (DNMT3A) antagonist, the insulin like growth factor 1 receptor (IGF1R) inhibitor, the mitogen-activated protein kinase 1 (MAPK1) inhibitor, the nitric oxide synthase 1 (NOS1) inhibitor, the glucocorticoid receptors (NR3C1) antagonist and the peptidylprolyl Isomerase A (PPIA) antagonist.

17. The method according to claim 16, wherein the first active agent is the retinoic acid receptor alpha (RARα) agonist, the second active agent is the voltage-gated potassium channel (KCNB2) inhibitor and the third active agent is the adrenergic receptor α2B (ADRA2B) antagonist.

18. The method according to claim 16, wherein the first active agent, the second active agent and the third active agent are administered simultaneously, separately or sequentially.

19. A combination of two or more active agents selected from the group consisting of a retinoic acid receptor alpha (RARα) agonist, a voltage-gated potassium channel (KCNB2) inhibitor, an adrenergic receptor α2B (ADRA2B) antagonist, a DNA methyltransferase 3 alpha (DNMT3A) antagonist, an insulin like growth factor 1 receptor (IGF1R) inhibitor, a mitogen-activated protein kinase 1 (MAPK1) inhibitor, a nitric oxide synthase 1 (NOS1) inhibitor, a glucocorticoid receptors (NR3C1) antagonist, and a peptidylprolyl Isomerase A (PPIA) antagonist; wherein the combination is used for the treatment of amyotrophic lateral sclerosis.

20. The combination according to claim 19, wherein:
the adrenergic receptor α2B (ADRA2B) antagonist is selected from mirtazapine and derivative thereof;
the retinoic acid receptor alpha (RARα) agonist is selected from acitretin and derivative thereof;
the voltage-gated potassium channel (KCNB2) inhibitor is selected from dalfampridine and derivative thereof;
the DNA methyltransferase 3 alpha (DNMT3A) antagonist is selected from azacitidine, decitabine and derivatives thereof;
the insulin like growth factor 1 receptor (IGF1R) inhibitor is selected from AXL-1717 and derivative thereof;
the mitogen-activated protein kinase 1 (MAPK1) inhibitor is selected from ulixertinib and derivative thereof;
the nitric oxide synthase 1 (NOS1) inhibitor is selected from ronopterin and derivative thereof;
the glucocorticoid receptors (NR3C1) antagonist is selected from mifepristones, ORG-34517 and derivatives thereof; and/or
the peptidylprolyl Isomerase A (PPIA) antagonist is selected from cyclosporine and derivative thereof.

21-28. (canceled)

29. The combination according to claim 19, wherein the combination comprises a first active agent and a second active agent, wherein the first active agent and the second active agent are different from each other and are independently selected from the group consisting of the retinoic acid receptor alpha (RARα) agonist, the voltage-gated potassium channel (KCNB2) inhibitor, the adrenergic receptor α2B (ADRA2B) antagonist, the DNA methyltransferase 3 alpha (DNMT3A) antagonist, the insulin like growth factor 1 receptor (IGF1R) inhibitor, the mitogen-activated protein kinase 1 (MAPK1) inhibitor, the nitric oxide synthase 1 (NOS1) inhibitor, the glucocorticoid receptors (NR3C1) antagonist and the peptidylprolyl Isomerase A (PPIA) antagonist.

30. The combination according to claim 29, wherein:
the first active agent is the retinoic acid receptor alpha (RARα) agonist and the second active agent is the adrenergic receptor α2B (ADRA2B) antagonist;
the first active agent is the voltage-gated potassium channel (KCNB2) inhibitor and the second active agent is the adrenergic receptor α2B (ADRA2B) antagonist; or
the first active agent is the retinoic acid receptor alpha (RARα) agonist and the second active agent is the voltage-gated potassium channel (KCNB2) inhibitor.

31-32. (canceled)

33. The combination according to claim 19, wherein the combination comprises a first active agent, a second active agent and a third active agent, wherein the first active agent, the second active agent and the third active agent are different from each other and are independently selected from the group consisting of the retinoic acid receptor alpha (RARα) agonist, the voltage-gated potassium channel (KCNB2) inhibitor, the adrenergic receptor α2B (ADRA2B) antagonist, the DNA methyltransferase 3 alpha (DNMT3A) antagonist, the insulin like growth factor 1 receptor (IGF1R) inhibitor, the mitogen-activated protein kinase 1 (MAPK1) inhibitor, the nitric oxide synthase 1 (NOS1) inhibitor, the glucocorticoid receptors (NR3C1) antagonist and the peptidylprolyl Isomerase A (PPIA) antagonist.

34. The combination according to claim 33, wherein the first active agent is the retinoic acid receptor alpha (RARα) agonist, the second active agent is the voltage-gated potassium channel (KCNB2) inhibitor and the third active agent is the adrenergic receptor α2B (ADRA2B) antagonist.

35. A pharmaceutical composition comprising the combination of claim 19 and a pharmaceutically acceptable carrier, wherein the combination is used for the treatment of amyotrophic lateral sclerosis.

36. A kit comprising the combination of claim 19 and an instruction for use, wherein the instruction describes the use of the combination or the pharmaceutical composition for treating amyotrophic lateral sclerosis.

37. The kit according to claim 36, wherein the two or more active agents are contained in the same or separate containers.

38-69. (canceled)
